(19) United States
(12) Patent Application Publication
(10) Pub. No.: US 2011/0275842 A1
(43) Pub. Date: Nov. 10, 2011

(54) NANOSTRUCTURED CATALYST PELLETS, CATALYST SURFACE TREATMENT AND HIGHLY SELECTIVE CATALYST FOR ETHYLENE EPOXIDATION

(76) Inventor: Gar B. Hoflund, Gainesville, FL (US)

(21) Appl. No.: 13/103,204

(22) Filed: May 9, 2011

Related U.S. Application Data
(60) Provisional application No. 61/333,064, filed on May 10, 2010.

(51) Int. Cl. C07D 301/04; B01J 23/50; B01J 23/66; B01J 37/08; B01J 37/34; B01J 35/10; B01J 37/00

(52) U.S. Cl. 549/536; 502/100; 502/300; 502/347; 502/5; 502/348; 549/523

(57) ABSTRACT

Catalyst pellets with a high BET surface area can be formed from the compression of a submicron powder into the selected pellet shape, such as using a press that forms the pellet in a die. Catalysts of particular interest comprise a ceramic material with an elemental metal coating. A low temperature plasma treatment can be used to achieve desired surface modification. Catalysts are described that have high selectivities in ethylene epoxidation reactions run over long time periods. The improved catalysts are based upon catalyst materials, such as ytrria coated with silver, with high selectivities. High BET surface areas can be achieved by using a particulate ceramic support material.

### CROSS REFERENCE TO RELATED APPLICATIONS

This application claims priority to copending U.S. provisional patent application 61/333,064 filed on May 10, 2010 to Hoflund, entitled “Nanostructured Catalyst Pellets and Highly Selective Catalyst for Ethylene Epoxidation,” incorporated herein by reference.

### FIELD OF THE INVENTION

The invention relates to nanostructured catalyst pellets that provide for high catalytic activity and selectivity. The invention further relates to specific catalyst formulations that provide for high selectivity for ethylene epoxidation. In addition, the invention relates to plasma surface treatment of the catalyst materials.

### BACKGROUND OF THE INVENTION

Commercial catalysts play an extremely important role in the chemical industry since they are widely used in chemical processes for producing a wide range of chemical compositions. Catalysts make many reactions practical through by providing reasonable reaction rates and/or reaction selectivity. With rising raw material costs and energy costs, improvement of efficiencies in chemical reactions can have a significant impact across industries relying on the products of the catalyzed reactions.

Ethylene oxide is an important commercial chemical with annual production in the U.S. alone in 1994 at 6.78 billion pounds. Ethylene oxide (C₂H₄O) has a three member ring with two carbon atoms and an oxygen atom and is the simplest epoxide. Ethylene oxide gas is used directly for sterilization in the medical industry since the gas kills many microorganisms. Ethylene oxide is also used as an intermediate for the production of a range of compositions including, for example, ethylene glycol, glycol ethers and surfactants. In 1994, almost 90% of the ethylene oxide produced in the U.S. was converted to ethylene glycol, which is then used to make, for example, automobile antifreeze and polyester fibers. Commercial production of ethylene oxide generally uses ethylene as the starting material, which is then partially oxidized under suitable conditions.

### SUMMARY OF THE INVENTION

In a first aspect, the invention relates to a catalyst pellet comprising a fused particulate material that comprises a ceramic material, and the particulate material can have a primary particle diameter of no more than about 250 nm. The catalyst pellet can have a BET surface area of at least about 5 m²/g. The density of catalyst pellet can be about 5 percent to about 90 percent of the density of the bulk density. In some embodiments, the catalyst pellet comprise a porous structure with pores having an average diameter from about 1 nm to about 900 nm. In some embodiments the catalyst pellet can further comprise about 5 weight percent to about 80 weight percent silver as an elemental coating.

In a further aspect, the invention relates to a catalyst pellet comprising a fused particulate material that comprises an elemental metal, the particulate material can have a primary particle diameter of no more than about 250 nm. The catalyst pellet can have a BET surface area of at least about 5 m²/g. The density of the catalyst pellet can be about 5 percent to about 90 percent of the density of the bulk density. The elemental mental of the catalyst pellet can comprise silver. In some embodiments, the catalyst pellet can have length and an orthogonal width. The length is no more than about twice the width.

In another aspect, the invention relates to a method for forming a nanostructured catalyst pellet, the method comprising pressing in a die a powder comprising a ceramic material with an average primary particle diameter of no more than about 250 nm, an elemental metal or combination thereof. The pressing in the die can be at a pressure sufficient to fuse the powder into a nanostructured pellet in the shape of the die. The pellet can have a BET surface area of at least about 5 m²/g. In some embodiments, the pressing can comprise applying a pressure from about 1000 psi to about 15,000 psi to the powder of particles. In some embodiments, the method can further comprise heating the particles at a temperature from about 350° C. to about 1000° C. The pellet can be heated for about 2 hours to about 24 hours. In other aspects, the invention relates to a method of preparing a catalyst material, the method comprising exposing a material comprising a ceramic material to a low temperature plasma to form a surface treated material. The method also comprises depositing elemental metal onto the ceramic material. The low temperature plasma of the method can be an oxygen plasma or a hydrogen plasma. The low temperature plasma can be applied for at least about 10 minutes. In some embodiments, the elemental metal can be silver or an alloy thereof. The ceramic material can comprise yttria. In some embodiments, the exposing to the low temperature plasma is performed before depositing the elemental metal onto the ceramic material. The exposing to the low temperature plasma can be performed both before and after depositing the elemental metal onto the ceramic material.

In another aspect, the invention relates to a catalyst material comprising at least about 10 weight percent elemental silver and at least about 10 weight percent yttria. In some embodiments, the catalyst material has at least 20 weight percent elemental silver. The catalyst can be particulate with particles having an average primary particle diameter of no more than about 250 nm. In general, the catalyst can comprise yttria with surfaces coated with the elemental silver. The catalyst material can have a BET surface area from about 1 m²/g to about 150 m²/g. The catalyst material can further comprises a dopant promoter comprising an alkali metal, such as Cs. In some embodiments, the dopant promoter concentration is at least 50 ppm by weight.

In additional aspects, the invention relates to a method for forming ethylene oxide from ethylene, the method comprising contacting ethylene with a catalyst in an atmosphere comprising oxygen, wherein the catalyst has a surface area from about 1 m²/g to about 150 m²/g. The reaction can have a selectivity from about 92% to about 100%. In some embodiments, the reaction has a conversion activity of at least about 5% at about 300° C. In some embodiments, the reaction is performed at a temperature of no more than about 300° C. The contacting of ethylene with the catalyst can comprise flowing the ethylene and the oxygen over the catalyst that is fixed in a reactor. The catalyst can comprise at least about 10 weight percent elemental silver and at least about 10 weight percent yttria. In some embodiments, the catalyst has a BET surface area from about 5 m²/g to about 60 m²/g. In some embodiments, the reactant selectivity is observed over a period of at least about 2 days.

### BRIEF DESCRIPTION OF THE DRAWINGS

FIG. 1 is a graph containing plots of conversion and selectivity versus time for a catalyst comprising 400 ppm Cs.

FIG. 2 is a graph containing plots of conversion and selectivity versus time for a catalyst comprising 400 ppm Cs where the catalyst was plasma treated after deposition of the surface active metal.

FIG. 3 is a graph containing plots of conversion and selectivity versus time for a catalyst comprising 750 ppm Cs.

FIG. 4 is a graph containing plots of conversion and selectivity versus time for a catalyst comprising 750 ppm Cs where the catalyst was plasma treated after deposition of the surface active metal.

FIG. 5 is a graph containing plots of the conversion and selectivity versus time for a catalyst comprising 750 ppm Cs and 100 ppm Re.

FIG. 6 is a graph containing plots of the conversion and selectivity versus time at 200 psig and at 230° C.

FIG. 7 is a graph containing plots of catalyst conversion and selectivity versus time demonstrating catalyst performance in an ethylene epoxidation reaction of 60 days.

FIG. 8 is a graph containing plots of catalyst conversion and selectivity versus time corresponding to an ethylene epoxidation reaction run at 200 psig and 230° C.

FIG. 9 is a graph containing plots of catalyst conversion and selectivity versus time corresponding to an ethylene epoxidation reaction run at 170 psig.

FIG. 10 is a graph containing plots of catalyst conversion and selectivity versus time corresponding to an ethylene epoxidation reaction run at two pressure ranges corresponding to different times of the run.

FIG. 11 is a graph containing plots of catalyst conversion and selectivity versus time corresponding to an ethylene epoxidation reaction run at three temperatures corresponding to different times of the run.

FIG. 12 is a graph containing plots of catalyst conversion and selectivity versus time corresponding to an ethylene epoxidation reaction run at 180° C., a pressure of 200 psig and a flow rate of 12.5 standard cubic centimeters per minute.

FIG. 13 is a graph containing plots of catalyst conversion and selectivity versus time corresponding to an ethylene epoxidation reaction run at three temperatures corresponding to different times of the run and with a reactant flow comprising a nitrogen carrier gas.

FIG. 14 is a graph containing plots of catalyst conversion and selectivity versus time corresponding to an ethylene epoxidation reaction run at three temperatures and pressures and with a reactant flow comprising a nitrogen carrier gas, in which the reaction was performed at a higher reactant concentration relative to the reactions used to generate the data plotted in FIG. 13.

FIG. 15 is a graph containing plots of catalyst conversion and selectivity versus time corresponding to an ethylene epoxidation reaction run with fragments of catalyst pellets and with a reactant flow comprising 17.6% ethylene and 7.3% oxygen.

FIG. 16 is a graph containing plots of catalyst conversion and selectivity versus time corresponding to an ethylene epoxidation reaction run with fragments of catalyst pellets and with a reactant flow comprising 28.4% ethylene and 11.7% oxygen.

### DETAILED DESCRIPTION OF THE INVENTION

Catalysts have been developed that provide significantly improved selectivity and activity for ethylene oxide synthesis through ethylene epoxidation. Improved selectivity results from the selection of a particularly appropriate combination of materials. In some embodiments, yttrium oxide, which can be referred to as yttria, is used as a support material for silver as the surface material of the catalyst. For ethylene epoxidation reactions, it has been found that the composition of the support material significantly affects the selectivity of the resulting catalyst. Appropriate engineering of the catalyst materials can be used to form catalysts with unprecedented selectivity for ethylene oxide synthesis. Thus, selectivities exceeding 92% for the epoxidation of ethylene have been achieved over extended periods of time. Furthermore, the catalyst materials can be formed into a nanostructured pellet that significantly improves the activity of the reaction. While the nanostructured pellets are specifically described with respect to ethylene oxide catalysts, nanostructured catalyst pellets can be adapted generally for the formation of inorganic catalysts. The catalyst pellets can be formed from submicron or nanoscale particles of the catalyst material. Under appropriate conditions the catalyst particles can be pressed in a die to form the nanostructured pellets. Generally, heat is applied to anneal the pellet while stabilizing the shape and size, and the heat can be applied in the die and/or after removal of the pellet from the die. Furthermore, catalyst materials with an elemental metal over a ceramic composition can be treated with a low temperature plasma to introduce desirable changes to the catalyst surface chemistry.

Based on the improved selectivity for ethylene oxide catalysts, a smaller fraction of the ethylene is lost as CO₂+H₂O oxidation products or other byproducts. The increased selectivity for ethylene oxide synthesis is significant with respect to the commercial cost and overall energy consumption. The potential for increased activity resulting from the high surface area of the catalyst can result in increased throughput of reactants for a quantity of catalyst per unit time, which can result in a decrease of capital expenses as well as a decreased use of catalyst for production of a particular amount of ethylene oxide or other catalyst product. The catalyst pellets can be formed in shapes convenient for handling and for use with commercial reactors.

Submicron particles, e.g., nanoparticles, are characterized by a very high BET surface area. Since the catalyzed reactions generally takes place on the catalyst surface, an increase in surface area can increase the conversion activity. To achieve desired levels of surface area, the primary particles can have in some embodiments an average particle diameter of no more than about 250 nm. The high surface area of the particles can contribute to desirable large reaction rates and correspondingly large throughput of reactants for a given reactor and total weight of catalyst. Submicron particles, however, can be difficult to handle as a catalyst in a commercial reactor. For example, in a flowing bed reactor, the submicron particles can be difficult to contain due to their small mass. It has been discovered that some of the submicron particle advantage can be maintained while improving catalyst handling. In particular, the submicron particles can be pressed to form a nanostructured pellet that still provides a large surface area. The pellets generally have a BET surface area of at least about 1 m²/g. The pellets can have reasonable sizes and shapes.

It has been discovered that the catalyst pellets can be formed by compressing submicron particles within a die with a press at an appropriate pressure to fuse the particles into the shape of the die without collapsing all of the nanostructure corresponding to the original submicron particles. Heat can be applied to anneal the pellet during and/or after the application of pressure. The selected temperature for the anneal step can be selected based on the properties of the particular materials. Thus, the resulting pellet can be nanostructured with a high surface area. Generally, after the formation of the pellets, some remnants of the original submicron particles can be visible on a micrograph of the pellet. The processing for the production of the pellets can be adjusted to yield a desired balance between mechanical strength of the resulting pellet and a high surface area. A press and die procedure is basically a molding under pressure in the die, and any equivalent process and apparatus referred to under a different terminology would be considered a press and die procedure.

Improved nanostructured catalyst pellets generally can be used for forming other heterogeneous catalysts besides ethylene oxide catalysts. In general, the particles comprise a ceramic material. The ceramic particles can be formed into the nano-structured ceramic pellets using the processes as described herein. If the ceramic material can function itself as a catalyst surface, the ceramic nanostructured pellets can then be used as the catalysts. If a metallic surface is desired to function as the catalyst surface, the nano-structured ceramic pellets can then be impregnated with metal to form the metal coatings. In alternative or additional embodiments, the ceramic submicron particles can be coated with an elemental metal or alloy prior to formation of the nanostructured pellet. In some embodiments, the submicron particles are coated with silver or other elemental metal or alloy prior to formation of the nanostructured pellets. Since elemental metals and alloys generally are relatively soft and malleable, the metal coated ceramic submicron particles can be conveniently formed into the nanostructured pellets. In further embodiments, metal or alloy submicron particles can be directly formed into nanostructured pellets without the need for a ceramic support.

In general, the improved ethylene oxide catalysts can comprise an yttria support that makes up a substantial portion of the mass of the catalyst. Silver coated onto the yttria forms the catalyst surface. The catalyst generally comprises at least about 5 weight percent silver and at least about 10 weight percent yttria, based on the total catalyst weight. The catalyst can further comprise an additive metal or metals that promote the catalyst activity. The optional promoter metal or metals can be present in amounts of at least about 10 ppm by weight based on the total catalyst weight. While it is preferred to form the ethylene oxide catalyst as a nanostructured pellet, the ethylene oxide catalyst material can be formed in any suitable structure. The support material and/or the silver coated material can be surface treated to modify the surface properties. In principle, the silver based catalysts can be used for other catalyzed reactions besides ethylene epoxidation if the other reactions can effectively use a supported silver catalyst.

A low temperature plasma treatment can be used to alter surface chemistries for catalysts comprising a ceramic support with an elemental metal coating on the support. In particular, a low temperature oxygen plasma can be desirable to remove surface hydrogen, but other low temperature plasma, such as a hydrogen plasma can be desirable. Commercial plasma generators can be adapted or the provision of such treatments. The low temperature plasma treatment for purely ceramic catalysts is described in U.S. Pat. No. 7,189,675 to Nagy, entitled “Olefin Polymerization Catalyst on Plasma-Contacted Support,” incorporated herein by reference. For catalysts comprising a ceramic carrier and a metal coating, the low temperature plasma treatment can be applied to the ceramic material prior to application of the metal coating and/or after the application of the metal coating. While the nano-particle and nano-structures catalyst materials described herein can be desirable materials, the low temperature plasma treatments can be applied for other structures of ceramic carriers with an elemental metal coating.

The catalyst material can be used within appropriate flow reactors for the epoxidation reactions. A blend of ethylene, oxygen and an optional diluent gas can be flowed through the system under pressure and heat. The improved catalyst materials have been able to achieve significantly improved selectivity for ethylene epoxidation. In particular, reaction yields of at least about 92% can be achieved. The high selectivity can be maintained over long periods of time. Improved yields result in a decrease of carbon dioxide production and a decrease in waste of ethylene. With the rising cost of energy and concern over carbon dioxide contribution to global warming, improvements in reaction yields for ethylene oxide production decreases carbon dioxide production as well as reduce cost through a cut of energy waste.

In summary, improved heterogeneous catalyst formats can be used for the production of high surface area catalysts that can be handled in a convenient way for appropriate reactions. With respect to embodiments based on the nanostructured catalyst pellets, as a result of the high surface area of the catalyst pellet, a greater throughput of reaction products can be achieved for a given weight of catalyst. This increased catalytic activity can result in a decreased cost for catalyst as well as a decrease in capital equipment costs since a greater amount of product can be produced with a particular weight of catalyst in a specific reactor. For ethylene oxide production, improved catalysts have been developed that provide for significantly improved selectivity of the reaction. The significant improvement in the selectivity provides a commercial advantage with respect to reduced waste and reduced carbon dioxide production.

### Catalyst Pellet and Methods to Form Catalyst Pellets

Desirable nanostructured catalyst pellets have been developed that can be adapted for the formation of a range of catalyst compositions. The pellets can comprise a ceramic composition, elemental metal or alloy and/or combinations thereof. The pellets are formed from submicron particles that are converted to the pellet structure without loss of all of the small particle-character of the original submicron particles, e.g., nanoparticles. The pellets can be formed, for example, by applying a selected amount of heat and pressure to form the pellets with a desired range of mechanical strength and surface area. The shape and size of the pellets can be selected for convenient use in an appropriate reactor.

The catalyst pellets generally comprise a ceramic material and/or an elemental metal as a coating over the ceramic material. Suitable ceramic materials include, for example, metal oxides, metal nitrides, metal carbides, metal sulfides, composites thereof, such as metal oxynitrides, and combinations thereof. As used herein unless otherwise noted, metals can refer to transition metals and non-transition metals as well as metalloids silicon, boron, germanium, arsenic, antimony, tellurium and polonium. Elemental metal coatings generally comprise an elemental metal or alloy thereof in which the metal or alloy is substantially in its unoxidized or elemental form. Elemental silver metal can possibly be a useful catalytic metal for selected applications including the ethylene epoxidation reaction described further below. In general, particular elemental metals can be useful as catalyst components for certain reactions, and platinum, palladium, ruthenium, rhodium and combinations thereof are generally useful in catalysts for a range of commercially significant reactions.

The word “element” is used herein in its conventional way as referring to a member of the periodic table in which the element has the appropriate oxidation state if the element is in a composition and in which the element is in its elemental form, M⁰, only when stated to be in an elemental form. Therefore, a metal element generally is only in a metallic state in its elemental form or a corresponding alloy of the metal's elemental form. In other words, a metal oxide or other metal composition, other than metal alloys, generally is not metallic. We note that for particulate materials and coated materials, the surfaces generally present different chemistries from the bulk. For example, a coating may be bonded to the underlying material, and/or the surface may present reorganizations due to dangling bonds, termination of crystal structures or the like as well as other surface modifications and bonding. Unless noted otherwise, references to compositions refer to the bulk material excluding surfaces between materials or along the outer surface or a particle.

For embodiments comprising both a ceramic support material and an elemental metal, the catalyst pellets can comprise generally from about 5 weight percent to about 80 weight percent metal, in further embodiments from about 8 weight percent to about 75 weight percent, and in additional embodiments form about 10 weight percent to about 70 weight percent elemental metal. The catalyst pellets can further comprise one or more additive metals that are present in promoter levels no more than about 2 weight percent that can provide desirable modification of the catalytic activity. Specific metal additives for the ethylene epoxidation reaction are described further below. Additive metals can be present in amounts from about 5 parts per million (ppm) to about 2000 ppm, in further embodiments from about 10 ppm to about 1500 ppm and in additional embodiments form about 20 ppm to about 1000 ppm. Since the metal additives are in low amounts the chemical form of the additive metals may not be clear. For example, the additive metals may be elemental of a metal composition. A ceramic material generally comprises the remaining portion of the catalyst pellet. A person of ordinary skill in the art will recognize that additional ranges of compositions within the explicit ranges above are contemplated and are within the present disclosure.

In embodiments of particular interest, the catalyst pellets are nanostructured. This can be evaluated in part through a determination of the surface area of the pellets. Surface areas can be evaluated using the BET surface area. BET (Brunauer, Emmitt and Teller) surface areas are based on adsorption of gases on the surface or the material. The BET method is described in Brunauer, et al., J. Am. Chemical Society, Vol. 60, pp 309-316 (1938). The measurement of BET surface areas is well established in the art. The catalyst pellets generally can have BET surface areas from about 1 m²/g to about 1000 m²/g, in further embodiments from about 2.5 m²/g to about 150 m²/g and in additional embodiments from about 5 m²/g to about 100 m²/g. For the ethylene epoxidation reactions described below, a very large BET surface area can result in a decrease of selectivity, so for these embodiments, the pellet can have a BET surface area from about 1 m²/g to about 125 m²/g, in further embodiments from about 2.5 m²/g to about 100 m²/g and in additional embodiments from about 5 m²/g to about 60 m²/g. A person of ordinary skill in the art will recognize that additional ranges of surface areas within the explicit ranges above are contemplated and are within the present disclosure.

To maintain the high surface areas observed for the pellets, the catalyst pellets maintain some of the structure of the submicron particles, e.g., nanoparticles, used to form the pellets. The submicron particles are fused into the structure of the pellets, but some character of the submicron particles remains. The degree of collapse of the submicron particle characteristics depends on the processing parameters. Generally, the mechanical strength of the pellets can be increased if some of the reduction of the surface area is sacrificed in the product pellet. However, in some embodiments, the characteristic particle sizes of the primary particles of the original submicron particles formed into the pellets can be observed in a transmission electron micrograph of the catalyst pellets. This visible structure can be reflective of the basic nanostructure of the pellets. The term nanostructured is intended to reflect the porous nature of the pellet as a result of the use of submicron particle, e.g., nanoparticles, in the formation of the pellet, and the specific ranges of the characterizing properties are discussed further below.

The pellets can be characterized through mechanical strength. The pellets can be characterized by crush strength, which can be performed with commercial measuring equipment using standardized procedures. However, a simpler test can be performed to indirectly evaluate mechanical strength using a shatter test in which a pellet is dropped a selected height onto a cement floor to determine if the pellets shatter upon impact.

While the catalyst pellets are nanostructured, the pellets have an overall structure that can be selected for convenient use in a reactor. The size and shape of the pellets can be characterized by the macroscopic dimensions of the pellet, which is evaluated based on assuming that the pellet is non-porous. In some embodiments, the pellets can be characterized by bulk physical dimensions, such as a length and width. In some embodiments, the characteristic length can be no more than a factor of two greater than the characteristic width and in further embodiments no more than a factor of about 1.5 times the characteristic width. In some embodiments, the length and width are approximately equal, such as for pellets that are roughly spherical. In some embodiments, the characteristic widths of the pellets can be from about 1/32 of an inch to about 1 inch, in further embodiments from about 0.05 ( 3/64) inch to about 0.75 inch and in other embodiments from about 0.075 inch to about 0.0625 ( 1/16) inch to about 0.65 inch, although specific dimensions can be selected for convenience for a particular reactor design. A person of ordinary skill in the art will recognize that additional ranges of characteristic bulk dimensions within the explicit ranges above are contemplated and are within the present disclosure. The shape of the pellets can be selected for convenience with respect to manufacturing as well as use in a reactor. While much of the BET high surface area of the catalyst pellets is associated with the internal nanostructure of the material, the shape of the pellets can be selected to have a relatively large bulk surface area, i.e., the surface area of a structure corresponding to the pellet with internal structure or porosity removed. In some embodiments, the pellets generally can have a desired shape, such as cylindrical or spherical. One pellet shape of particular interest is a cylindrical shape with an open core. Cylindrical catalyst elements have been used with some traditional alumina supported catalysts, as described in U.S. Pat. No. 7,547,795 to Matisz et al., entitled “Silver-Containing Catalysts, the Manufacture of Such Silver-Containing Catalysts, and the Use Thereof,” incorporated herein by reference.

The porosity of the pellets can be evaluated in several different ways. As noted above, the BET surface area reflects some features of the porosity, and the BET surface area generally reflects the working surface area of the catalysis reaction. Other ways to evaluate the porosity include the density of the pellets, which can be expressed as a fraction of the bulk density based on the composition of the pellet. For example, if the pellet comprises 50 weight percent yttria and 50 weight percent silver metal, the bulk density for the pellet composition would be the average of the bulk density of yttria and silver. In some embodiments, the pellet can have a density from about 5 percent to about 90 percent of the bulk density, in further embodiments from about 7.5 percent to about 85 percent and in other embodiments from about 10 percent to about 80 percent of the bulk density. A person of ordinary skill in the art will recognize that additional ranges within the explicit ranges of density above are contemplated and are within the present disclosure.

Also, the porosity can be evaluated from visual inspection of the surface using micrographs. In particular, an area of the surface can be evaluated with respect to the portion of the area covered by pores. From a micrograph of pellets, the area covered by pores can be evaluated relative to the total area along the surface of the pellet. The porosity of the particles can be evaluated using mercury porosimetry, which can be based on corresponding commercial measuring equipment. In general, the pores can comprise from about 5 percent to about 75 percent of the pellet volume, in further embodiments, from about 7.5 percent to about 65 percent and in additional embodiments from about 10 percent to about 60 percent of the pellet volume. Furthermore, it has been observed that the pores have a bimodal distribution of sizes. Observed nanopores can have an average pore diameter from about 1 nanometers (nm) to about 15 nm, in further embodiments from about 1.5 nm to about 10 nm and in additional embodiments from about 2.0 nm to about 7.5 nm. Observed macropores can have an average pore diameter of about 100 nm to about 1.5 microns, in further embodiments from about 150 nm to about 1 micron and in additional embodiments from about 200 nm to about 900 nm. A person of ordinary skill in the art will recognize that additional ranges of pore parameters within the explicit ranges above are contemplated and are within the present disclosure.

In some embodiments, if the pellets comprise an elemental metal active surface on a ceramic support, the pellets can be formed before or after the metal is associated with the support material. The methods for forming the pellets are described below. However, if the metal is coated onto the ceramic submicron particles prior to forming the pellet, the metal can be more uniformly coated over the ceramic support throughout the nanostructured pellet, which may provide improved catalytic performance. Furthermore, the metal surfaces can provide for more effective pellet formation under appropriate formation conditions due to the malleable nature of most elemental metals, which can be reflected in the softening temperature of the metal. Therefore, in some embodiments it is desirable use elemental metal coated ceramic particles, such as submicron particles, to form the pellets.

In general, the metal can be coated onto ceramic submicron particles using any appropriate process. Impregnation based approaches are desirable in which the metal is reduced in contact of the ceramic submicron particles such that that metal is formed as a coating onto the ceramic submicron particles. The solution based deposition of metals is described further below with respect to the formation of ethylene oxide catalysts.

As noted above, the pellets can comprise an elemental metal without a ceramic support. For example, for the ethylene epoxidation reactions described further below, the pellets can be formed from elemental silver particles that are processed directly into the pellets. Good catalytic performance have been observed with such pellets, although the cost is generally high for such pellets since silver is relatively expensive. A promoter metal can be included in low levels as described below. For example, a cesium nitrate solution can be mixed with the silver particles and then the liquid is removed through evaporation to introduce, for example, 500 ppm by weight Cs.

The submicron particle precursors to the pellets generally have an average primary particle diameter of no more than about 250 nm, in further embodiments from about 2 nm to about 150 nm and in additional embodiments from about 3 nm to about 100 nm. A person of ordinary skill in the art will recognize that additional ranges of average primary particle diameters within the explicit ranges above are contemplated and are within the present disclosure. The particle diameters can be evaluated as an average diameter for non-spherical particles. Particle diameters are evaluated from an inspection of transmission electron micrographs in which the primary particles are the visible grains in the micrograph. Suitable ceramic nanoparticles are available from commercial sources or can be prepared by several methods.

The pellets can be formed by adapting commercial press and die processes. A powder of the precursor nanoparticles can be supplied into the die. In general, the nanoparticles can be elemental metal particles, ceramic particles of elemental metal coated ceramic particles. Then, the press is engaged to apply pressure to the material in the die. The die has an appropriate inner shape to form the selected pellet shape. Heat generally can be simultaneously applied with pressure and/or after the pressure treatment to fix the pressed structure. Suitable pressures generally range from about 1000 psi to about 15,000 psi, in further embodiments from about 1500 psi to about 12,000 psi and in additional embodiments from about 2000 psi to about 10,000 psi. Suitable temperatures for annealing the pellet generally range from about 350° C. to about 4500° C., in further embodiments from about 375° C. to about 2000° C., and in additional embodiments from about 400° C. to about 1000° C. The selection of temperature generally depends on the melting point and corresponding softening temperatures for the materials involved. For example, for the silver based catalysts described herein, the processing temperatures would generally be under the silver melting point of about 962° C. Similarly, the amount of time for the delivery of the pressure or temperature can be adjusted similarly based on the materials. The heat can be applied for a period from about 2 hours to about 24 hours, in further embodiments from about 3 hours to about 12 hours and in other embodiments from about 4 hours to about 8 hours, along with appropriate ramp up and ramp down times. A person of ordinary skill in the art will recognize that additional ranges of pressure and temperature within the explicit ranges above are contemplated and are within the present disclosure. For a specific pellet composition and desired pellet properties, the specific pressure and temperature can be selected. In general, higher pressures and temperatures result in a lower surface area and greater mechanical strength of the pellet, and correspondingly lower pressures and lower temperatures result in higher surface areas and lower mechanical strengths. The temperature and pressure can be selected based on the teachings herein to obtain the desired balance of mechanical strength and surface area.

Commercial press and die systems are commercially available. Suitable presses are available, for example, from Carver, Inc. (Wabash, Ind.), Across International (New Providence, N.J.), SPEX Sample Prep (Metuchen, N.J.), Specac Ltd. (Cranston, R.I.) and Reflex Analytical Corporation (Ridgewood, N.J.). The dies are generally machined to selected specifications to match the press and pellet parameters. The process of filing the die and extracting the pellet can be automated for commercial production.

In addition or as an alternative to the use of pressure and heat, the pellet can be formed using a burnout material. For example, the nanoparticles can be combined with a burnout material as a matrix for a composite precursor composition. The composite precursor composition can be pressed or molded into a desired shape. The burnout material can be combusted in an oxygen containing environment to remove the burnout material as product gases from the combustion. The burnout material contributes to the pore formation, and the use of burnout material can provide additional control to the pore formation relative to the use of pressure and heat without the use of the burnout material.

Suitable burnout materials include, for example, carbon particles, such as carbon blacks and graphite, corn starch, organic polymers, such as vinyl polymers, or the like. The burnout materials can be blended with the catalyst particles to form a uniform composite blend. The heat generated by the burnout of the burnout material can further contribute to the fusing of the catalyst particles to form a stable nanostructured pellet with appropriate mechanical strength. Air, oxygen or other oxygenated gas can be flowed over the pellets as the burnout material is being combusted.

### Low Temperature Plasma Treatment of Metal Coated Ceramic Catalysts

It can be desirable to treat the catalyst with a plasma during the preparation of the catalyst for an appropriate duration. In general, the plasma can be contacted with the catalyst support prior to deposition of an elemental metal surface material and/or with the catalyst after deposition of the elemental metal surface material. Without being limited by a theory, it is believed that plasma treatment of the catalyst can help prevent deactivation and/or help promote activation of the catalyst material during reaction. As used herein, the term ‘plasma’ refers to an energized gas comprising positively charged ions, electrons, and neutral particles.

The plasma can be generated by energizing a precursor gas. In general, processes for the formation of a low temperature plasma can include, for example, applying a voltage across the precursor gas or irradiating the precursor gas with electromagnetic radiation. Suitable precursor gasses can include, for example, air, argon, helium, hydrogen, neon, nitrogen, oxygen, xenon and combinations thereof. For ethylene oxide catalysts, particularly desirable precursor gases include, for example, hydrogen and oxygen for the respective formation of a hydrogen or oxygen plasma. A method for plasma treatment using microwaves as an energizing means is discussed in U.S. Pat. No. 7,189,675 to Nagy, entitled “Olefin Polymerization Catalyst on Plasma-Contacted Support,” incorporated herein by reference. A method for plasma treatment using electromagnetic fields generated by the application of RF power is described in U.S. Pat. No. 3,485,771 to Horvath, entitled “Plasma Activation of Catalysts,” also incorporated herein by reference.

The low temperature plasma treatments generally can be performed effectively at low pressures, such as pressures less than about 10 Torr and in some embodiments no more than about 5 Torr, and lower pressures can be used. In some embodiments, the treatment is performed in a high vacuum chamber that is evacuated to very low pressures before backfilling to a desired pressure of the gas that is used to form the plasma. If desired the formation of a plasma can be determined through the visual observation of a glow discharge. For an oxygen plasma in the pressure ranges described herein, voltages on the order of 1000 volts with an oxygen pressure on the order of 1 Torr or less can be used to generate the plasma, and a person of ordinary skill in the art can adjust the plasma parameters based on the teachings herein. The plasma is generally applied for at least about 5 minutes, in further embodiments at least about 10 minutes, and the plasma can be applied for 15 minutes to a day or more. A person of ordinary skill in the art will recognize that additional ranges of plasma parameters within the explicit ranges herein are contemplated and are within the present disclosure.

The catalyst and plasma can be contacted so as to promote exposure of the catalyst particles to the plasma. An apparatus for plasma treatment using a directed plasma flow generated in the presence of a catalyst is discussed in U.S. Pat. No. 3,485,771 to Horvath, entitled “Plasma Activation of Catalysts,” incorporated herein by reference. An apparatus for low temperature plasma treatment suitable for larger quantities of particles is described in U.S. Pat. No. 5,278,384 to Matsuzawa et al., entitled “Apparatus and Process for the Treatment of Powder Particles for Modifying the Surface Properties of the Individual Particles,” incorporated herein by reference. Commercial suppliers of plasma treatment systems include, for example, Plasmatreat US LP, Elgin, Ill., Enercon Industries Corporation, Menomonee Falls, Wis. and Plasma Etch, Inc., Carson City, Nev.

### Ethylene Epoxidation Catalysts

Generally, catalysts for the reaction of ethylene to form ethylene oxide are based on silver supported on a ceramic support material, such as α-alumina. The improved catalysts described herein involve improved properties that lead to significantly improved catalytic performance properties. The properties of the catalysts depend significantly on the parameters of the catalyst materials, and the selection of these properties significantly influences the efficacy of the resulting catalysts for ethylene epoxidation. Specifically, extremely good selectivity has been obtained through the design of the catalysts as described herein. Also, these high levels of selectivity have been obtained while still maintaining good conversion rates within test systems.

The industrial ethylene epoxidation catalysts generally comprise an α-alumina (alpha phase aluminum oxide) support material, a silver coating and an optional dopant metal. Specifically, commercial catalysts are generally formed with an α-alumina, support material that is microporous. See, for example, U.S. Pat. No. 5,008,413 to Liu, entitled “Catalyst for Oxidation of Ethylene to Ethylene Oxide,” (the Liu patent) and U.S. Pat. No. 4,242,235 to Cognion et al., entitled “Supports for Silver Catalysts Utilized in the Production of Ethylene Oxide,” both of which are incorporated herein by reference, which describe alumina and silica supports. It has been suggested that highly pure α-alumina was significant for obtaining better ethylene oxide selectivity, as described in published U.S. patent application 2009/0177000A to Natal et al., entitled “Alkylene Oxide Catalyst and Use Thereof,” incorporated herein by reference (the Natal application). As described herein, it has been discovered that yttrium oxide, i.e., yttria, is a superior support material for ethylene epoxidation reactions. Specifically, it has been found that catalysts formed with yttria support materials can result in superior selectivity.

It has been suggested that the nature of the support material can influence the performance of ethylene oxide catalysts. See, “Support Participation in Chemistry of Ethylene Oxidation on Silver Catalysts,” Lee et al., Applied Catalysts, Vol. 44 (1988) 223-237, incorporated herein by reference (hereinafter the Lee article). However, the results in the Lee article all resulted in relatively low selectivity, and the commercial relevance is not clear. These catalysts only included 2 weight percent silver as noted on page 226 of the Lee article. The Lee article reports that use of an ytrria support leads to no ethylene oxide production and a very low total rate. Thus, the Lee article teaches away from the use of yttria, and it is not completely clear why the Lee catalyst with yttria failed, although the low amounts of silver are noted above.

The improved ethylene epoxidation catalysts described herein comprise from about 5 weight percent to about 90 weight percent elemental silver, in further embodiments from about 10 weight percent to about 80 weight percent, in other embodiments from about 15 weight percent to about 70 weight percent and in additional embodiments from about 20 weight percent to about 60 weight percent. The catalysts generally also comprise a dopant metal or metals that enhance performance of the catalysts, which are described further below. In some embodiments, the catalyst comprises a dopant promoter in an amount from about 10 ppm by weight to about 1 weight percent, in further embodiments from about 50 ppm by weight to about 0.5 weight percent, in additional embodiments from about 75 ppm by weight to about 0.25 weight percent and in other embodiments from about 100 ppm by weight to about 0.1 weight percent (1000 ppm by weight). The remaining weight of the catalyst is generally substantially made up of the support material. A person of ordinary skill in the art will recognize that additional composition ranges within the explicit ranges above are contemplated and are within the present disclosure.

The dopant promoter metal element can be an alkali metal element. In particular potassium, rubidium and cesium have been identified as useful promoter metals. It is not known with certainty if the promoter metal is in elemental form or is present as a metal composition with the metal in an appropriate oxidation state. The use of alkali promoter additives and a summary of their historical use is described further in U.S. Pat. No. 5,691,269 to Rizkalla, entitled “Process for Preparing Silver Catalyst,” incorporated herein by reference. The use of thallium as an alternative to an alkali metal as an accelerator is described in U.S. Pat. No. 4,389,338 to Mitsuhata et al., entitled “:Method for Manufacture of Silver Catalyst for Production of Ethylene Oxide,” incorporated herein by reference. A broader range of promoters including K, Ca, Cs, Ba, Pt, Ni, Sn, Cd, Sr, Li, Mg, Na, Rb, Au, Cu, Zn, La, Ce, Th, Be, Sb, Bi, Ti, Pd, Ir, Os, Ru, Fe and Al has been described in U.S. Pat. No. 4,242,235 to Cognion et al., entitled “Supports for Silver Catalysts Utilized in the Production of Ethylene Oxide,” incorporated herein by reference. The Nadal application, above, suggests that a plurality of promoters can be beneficial and that certain anions can also function as promoters in addition to metals. The suggested anions included halogens and polyatomic oxyanions, such as sulfates, phosphates, titanates, tantalates, molybdates, vanadates, chromates, zirconates, polyphosphates, manganates, nitrates, chlorates, bromates, borates, silicates, carbonates, tungstates, thiosulfates, cerates and mixtures thereof. The metal additives generally can be added before, after and/or during the deposition of the elemental silver.

In some embodiments, the support material is in the form of submicron particles. The method for the synthesis of the yttria submicron particles is generally not significant. Suitable yttria submicron particles are available commercially. For example, yttria submicron particles can be obtained from Nanostructured and Amorphous Materials (Houston, Tex.), Inframat Advanced Materials (Manchester, Conn.), Sky Spring Nano Materials (Houston, Tex.) and Nanophase Technologies Corp. (Romeoville, Ill.). While the submicron particles can be used directly as catalysts as described in the examples, these submicron particles can be formed into pellets as described above. The silver is generally deposited on the particles prior to forming the pellet. A promoter metal or metals can be added before or after pellet formation.

The catalyst materials can be surface treated prior to the deposition of silver or after the deposition of silver to alter the surface chemistry. For example, the particle surfaces can be cleaned using chemical or mechanical cleaning processes. Also, the Liu patent teaches the heating of the support material to 85° C. for 30 minutes before depositing the silver. As described further below, the support material can be subjected to a plasma treatment prior to depositing the silver. For example, the plasma treatment can comprise treatment with a low temperature oxygen plasma treatment, comprising a flow of oxygen atoms or a low temperature hydrogen plasma treatment comprising a flow of hydrogen atoms.

The silver and promoting metals are contacted with the support yttria in the desired amounts followed by heating to reduce the metal. For silver, the heating can be performed in air, which provides an environment that is not excessively oxidizing. For example, silver nitrate can be used as a silver source.

Alternative approaches using a silver oxide reactant are described in U.S. Pat. No. 4,916,243 to Bhasin et al., entitled “Catalyst Compositions and Process for Oxidation of Ethylene to Ethylene Oxide,” incorporated herein by reference. For example, an aqueous lactic acid solution can be used to dissolve the silver oxide (Ag₂O) to form a precursor silver solution. Salts of promoter metal(s) can be similarly dissolved into this solution. A similar procedure to form the silver precursor solution is described in an article to Hoflund et al., entitled “Study of Cs-Promoted, α-Alumina-Supported Silver, Ethylene-Epoxidation Catalysts, II. Effects of Aging,” Journal of Catalysis 162, pp 48-53 (1996), incorporated herein by reference.

The solution is then contacted with the support and dried. To obtain the desired amount of silver in the catalyst materials efficiently and uniformly applied to the support material, the deposition process can be repeated a second, third or more times. To reduce the silver to silver metal, the dried support can then be heated for a sufficient period of time to reduce the silver at temperatures from about 100° C. to about 900° C., in some embodiments from about 250° C. to about 800° C., and in further embodiments from about 350° C. to about 600° C. In general, the materials are heated for at least about 30 seconds, in further embodiments from about 45 seconds to about 5 hours, and in other embodiments from about 1 minute to about 1 hour. The amount of time to reduce the silver may depend on how dry the material is initially. A person of ordinary skill in the art will recognize that additional ranges of temperatures and reduction times within the explicit ranges above are contemplated and are within the present disclosure.

### Ethylene Epoxidation Reaction

The reaction to form ethylene oxide form ethylene involves a flow of reactant gases over the catalyst under heated conditions. The composition of the flow gases are selected to provide desired results. Undesirable by-products include, for example, the complete oxidation products of water and carbon dioxide. The ability of the catalysts to direct the reaction to the desired ethylene oxide product is evaluated in terms of a quantity generally referred to as selectivity. The overall reaction of the ethylene can be separately described in terms of conversion of the ethylene. Any un-reacted ethylene can be removed from the product flow and recirculated to the reactor.

The reactions are generally carried out in a reactor with a packed catalyst bed. The catalyst is placed in the bed and the reactants are flowed through the bed. An example of an appropriate reactors are described in U.S. Pat. No. 4,177,169 to Rebsdat et al., entitled “Process for Improving the Activity of Used Supported Silver Catalysts,” and in U.S. Pat. No. 7,547,795 to Matusz et al., entitled “Silver-Containing Catalysts, the Manufacture of Such Silver-Containing Catalysts, and the Use Thereof,” both of which are incorporated herein by reference. The reactor generally comprises an elongated reactor tube holding the packed catalyst bed. The catalyst pellets or other catalyst structures are held within the packed catalyst bed. The reactor tube can be jacketed to allow a flow of a cooling fluid to control the temperature within the reactor tube. While the reaction is performed at elevated temperatures, the reaction is exothermic so that cooling water is generally needed to maintain the reactor at the desired temperature. In particular the undesired side reactions that result in further oxidation of the starting material are significantly more exothermic than the epoxidation reaction. Thus, the increase selectivity available as described herein can reduce the use of cooling fluid for thermal management. The tube can be fitted with appropriate connections to provide for the flow of reactants into the reactor tube and the flow of products out from the reactor tube under controlled conditions.

The parameters of the reaction in the packed bed reactor include, for example, the inlet pressure, the flow rate, composition of the flow and the temperature of the fluidized bed, i.e., catalyst. In general, the catalyst temperatures are from about 140° C. to about 450° C., in further embodiments from about 145° C. to about 400° C., and in further embodiments from about 150° C. to about 350° C. For commercial reactors, the inlet pressure generally ranges from about 150 psi (1034 kPa) to about 500 psi (3447 kPa), and in further embodiments from about 200 psi (1379 kPa) to about 400 psi (2758 kPa). A person of ordinary skill in the art will recognize that additional ranges of temperature and pressure within the explicit ranges above are contemplated and are within the present disclosure. Suitable flow rates generally depend on the particular reactor design. The improved catalysts described above can achieve their improved performance characteristics at relatively lower catalyst temperatures, which may lead to relatively longer catalysts lifetimes.

The reactant flow can use either air or purified oxygen as an oxygen source for the reaction with appropriate adjustments to obtain a desired oxygen concentration. One or more inert diluent or moderator gases generally can used in the flow, and suitable diluent or moderator gases generally include, for example, carbon dioxide, ethane, ethyl chloride, argon, helium, nitrogen gas (N₂) or combinations thereof. With the improved catalysts described herein, the flow may not comprise ethane as a diluent gas or ethylene chloride or other gaseous alkyl halides, such as ethyl chloride, moderator. It is desirable to avoid the use of alkyl halides, but these are generally used for conventional catalysts to slow the reaction to obtain longer life to the catalyst. With the present catalysts, damage to the catalyst generally can be avoided or reduced if ethylene chloride or other organic halide is not used in the flow. The flow generally comprises from about 1 mole percent to about 50 mole percent ethylene, in further embodiments from about 2.5 mole percent to about 45 mole percent and in additional embodiments form about 5 mole percent to about 40 mole percent ethylene. Also, the flow generally comprises from about 2 mole percent to about 15 mole percent oxygen, in further embodiments from about 2.5 mole percent to about 14 mole percent and in other embodiments from about 3 mole percent to about 12 mole percent oxygen. The remainder of the flow generally comprises inert diluent gases. A person of ordinary skill in the art will recognize that additional compositional ranges within the explicit ranges above are contemplated and are within the present disclosure.

The catalysts as described above provide superior performance in ethylene epoxidation reactions. In particular, the initial performance of the catalyst has been demonstrated as provided in the examples below. Of particular significance, the catalysts can provide superior selectivity. The selectivity is defined as 100×(moles of ethylene oxide formed/moles of ethylene reacted). The catalysts can exhibit initial as well as ongoing selectivities of at least about 92 percent, in further embodiments at least about 93 percent, in additional embodiments at least about 94 percent and in other embodiments from about 94.5 percent to about 99.5 percent, although approximately 100 percent conversion may be achieved. As described in the examples below, desired high selectivities have been achieved at both initial times as well as for longer times following initial transitory period. High selectivities can be achieved for periods of time of at least about two days, in further embodiments to 5 days and in additional embodiments for at least 10 days. A person of ordinary skill in the art will recognize that additional ranges of selectivities within the explicit ranges above are contemplated and are within the present disclosure. The high selectivities noted above have been obtained at low temperatures, specifically no more than about 200° C. It can be desirable to operate the reaction at lower temperatures to reduce energy use. However, in further embodiments, the reactions are performed at temperatures of no more than about 300° C. A person of ordinary skill in the art will recognize that additional temperature ranges within these explicit ranges are contemplated and are within the present disclosure.

The overall productivity of the reaction can be evaluated in different ways. For example, the overall rate of ethylene oxide production can be evaluated or the amount of ethylene oxide produced as a fraction of the ethylene reactant can be used as a measure of the production rate. More commonly, the percent of ethylene reactant that is reacted is evaluated as a percentage of the initial ethylene reactant. This can be referred to as the conversion, which is equal to the 100×(moles of ethylene before reaction−moles of ethylene after reaction)/moles of ethylene before reaction). Also, the conversion values may be scaled by the amount of catalyst. The conversion can be relatively temperature sensitive. There is a tradeoff since running the reaction at a greater temperature can result in greater conversion at the cost of a shorter catalyst lifetime.

### EXAMPLES

### Example 1

### Formation of Silver Supported Submicron Particle Catalysts

This example describes the formation of submicron catalyst particles comprising silver supported on a yttria support material with a Cs promoter metal.

Yttria submicron particles were obtained from Inframat Advanced Materials (Manchester, Conn.). The particles were catalog #39N-0802, 99.95% pure and with an average particle size of 30-50 nm. The supplier claimed a specific surface area of 30-50 m²/g from a multi-point BET analysis. A 0.02 wt % CsNO₃ stock solution was prepared, and a specific amount of this solution was added to deionized water to form a desired concentration. The diluted solution was heated to about 80° C., and a AgNO₃ precursor was dissolved into this solution. In some samples, a solution of Re₂O₇ was also added to the precursor solution. After the AgNO₃ was completely dissolved, the Y₂O₃ powder was added and mixed thoroughly. The water was allowed to evaporate under constant stirring for approximately 1 hour until all standing water was gone. The resulting catalyst was scraped from the sides of the container and broken into small pieces with a spatula. The container with the catalyst was then placed in an oven at 104° C. for approximately 20 hours to completely dry the catalyst.

The resulting catalyst had approximately 40 weight percent silver and between 400 ppm and 1000 ppm by weight Cs, with 750 ppm of Cs commonly used. Some selected samples also had between 40 ppm and 100 ppm by weight Re. Also, in the preparation of selected samples, the Y₂O₃ particles were subjected to a low-temperature oxygen plasma for 75 minutes prior to addition to the solution with the silver nitrate. In some other selected samples, the low-temperature plasma treatment was performed on the catalyst particles after silver deposition and drying of the catalyst. The low temperature plasma was applied to a thin layer of powder in a vacuum chamber backfilled with a low pressure of oxygen based on a 1000 volt status potential that generates the plasma. BET surface area measurements on a representative catalyst power yielded a surface area of 37.9 m²/g.

### Example 2

### Ethylene Epoxidation with Powdered Catalyst: Shorter Time Performance

This example demonstrates the superior shorter time performance of the catalyst materials described herein in ethylene epoxidation reactions. In particular, superior selectivity is achieved using the catalyst particles formed as described in Example 1.

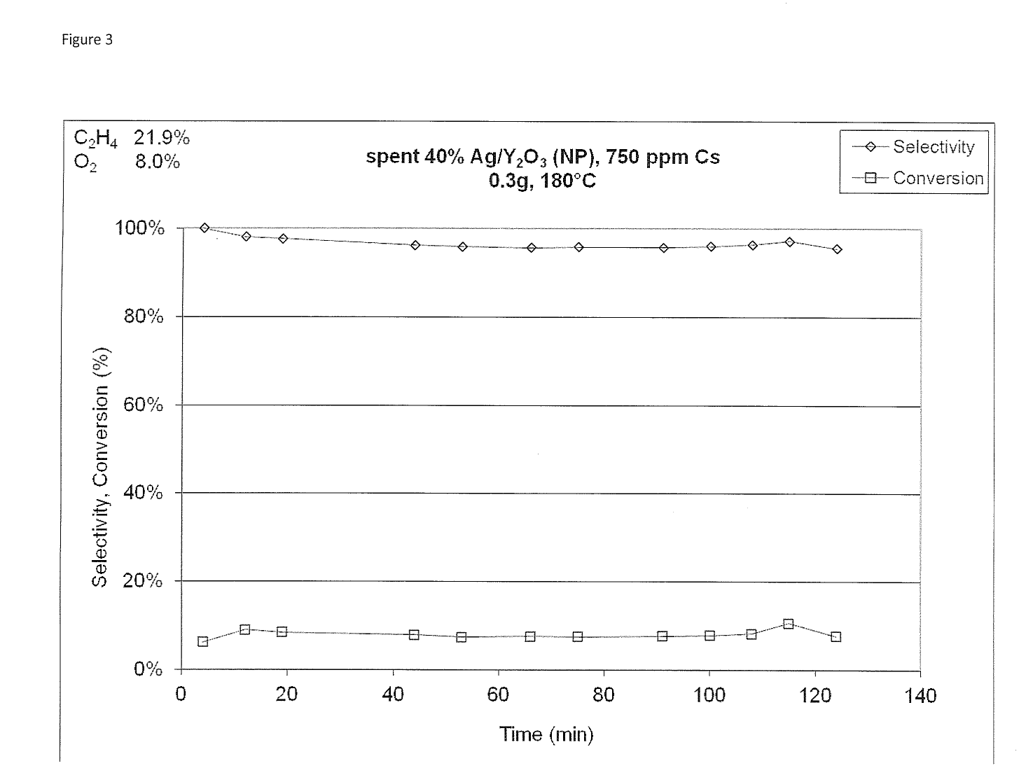

The reactions were performed using a glass tube extending through a furnace to heat the catalyst to the target temperature. The particles were held in place in the tube with glass wool and fittings were attached on both ends of the tube to control the flow through the tube. The gas consisted of 4-25 vol % (percent by volume) ethylene as specified more explicitly below and 8-10 vol % oxygen, the remainder of the gas being a carrier gas comprising helium. The catalyst powders were prepared as described in Example 1. Specific catalyst and reaction parameters are displayed in Table 1, below. Note that the catalyst powder used to obtain the results shown in FIG. 3 was a catalyst that had been previously used.

TABLE 1

| | | | | | Inlet Gas |
|---|---|---|---|---|---|
| | Promotor | Tempera- | | Plasma | Composition |
| | (Composition, | ture | Pressure | Treat- | (vol % C₂H₄, |
| Figure | Amount) | (° C.) | (psig) | ment | vol % O₂) |
| 1 | Cs, 400 ppm | 200 | 8-9 | None | 22.7, 8.0 |
| 2 | Cs, 400 ppm | 200 | 13 | After | 23.0, 8.0 |
| | | | | AgNO₃ | |
| | | | | addition | |
| 3 | Cs, 750 ppm | 180 | 8-9 | None | 21.9, 8.0 |
| 4 | Cs, 750 ppm | 200 | 8-9 | After | 22.9, 8.2 |
| | | | | AgNO₃ | |
| | | | | addition | |
| 5 | Cs, 750 ppm | 210 | 8-9 | None | 22.6, 8.8 |
| | and | | | | |
| | Re, 100 ppm | | | | |
| 6 | Cs, 750 ppm | 230 | 200 | After | 9.9, 4.9 |
| | | | | AgNO₃ | |
| | | | | addition | |

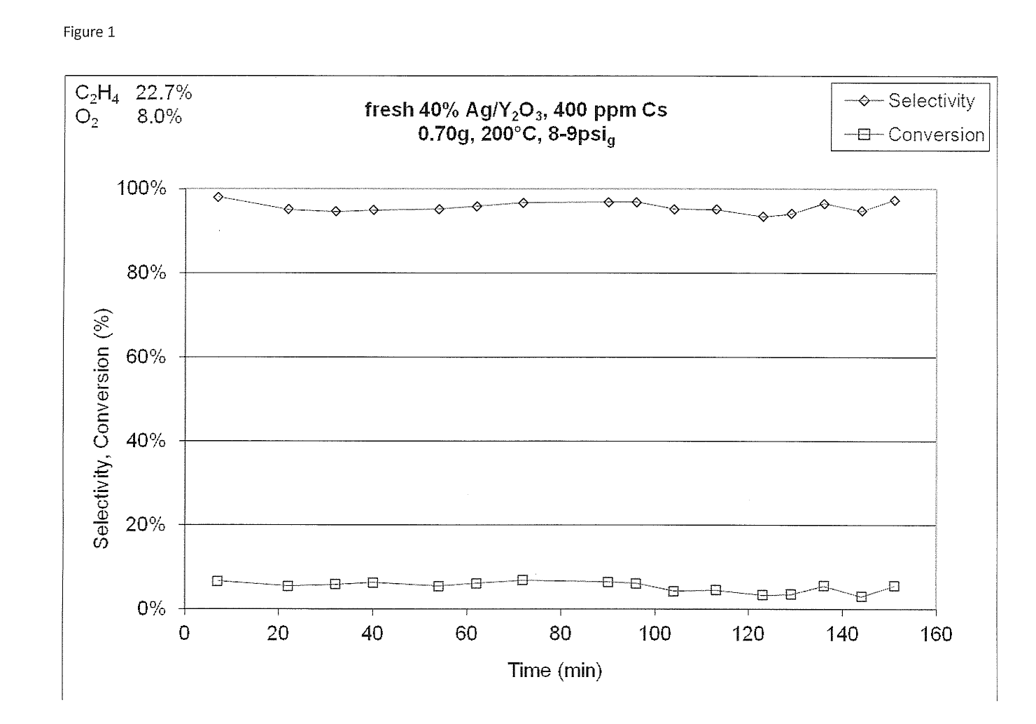

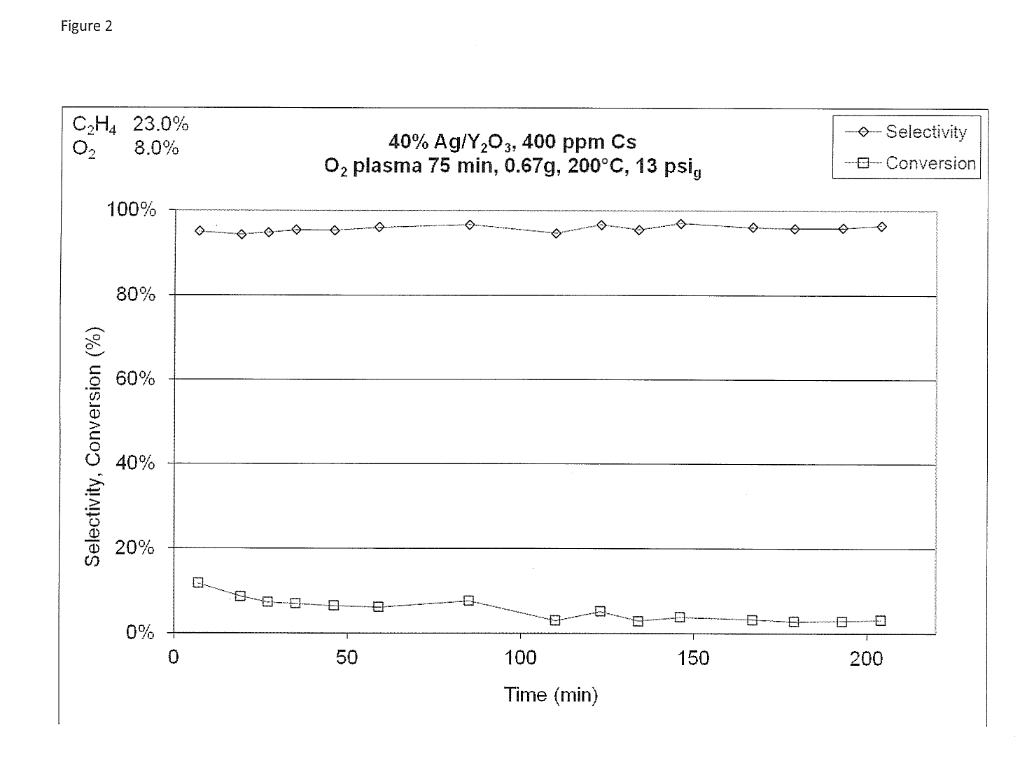

Referring to FIGS. 1 and 2, ethylene epoxidation results are presented for two runs with catalysts having 400 ppm by weight Cs at low pressures. The catalyst used in the epoxidation reaction corresponding to FIG. 2 was treated with an oxygen plasma for 75 minutes. Both of these runs demonstrated roughly 95% selectivity or greater and respectable conversions at a relatively low temperature.

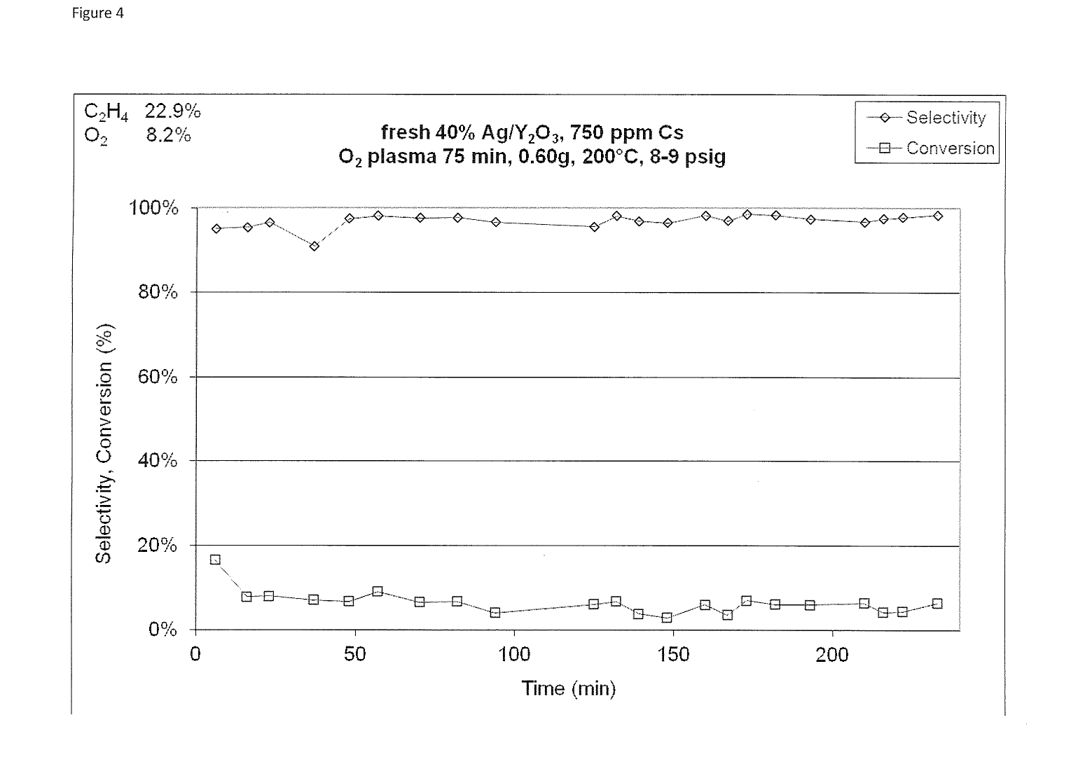

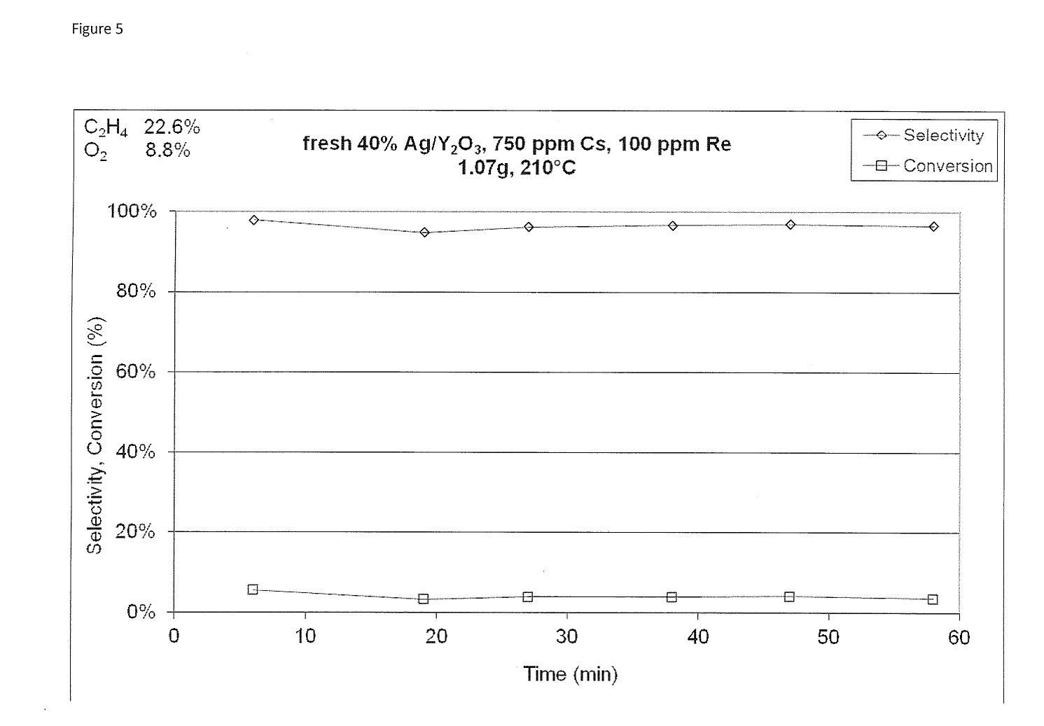

Referring to FIGS. 3 and 4, corresponding results are presented for catalysts having 750 ppm by weight Cs. The catalyst used in the epoxidation reaction corresponding to FIG. 4 was treated with an oxygen plasma for 75 minutes. The results demonstrated in FIGS. 3 and 4 demonstrate selectivity greater than 97%-98% over most of the time range. Again, the conversions were reasonable. Comparable results were also obtained with the addition of 100 ppm by weight Re in the catalyst, as shown in FIG. 5.

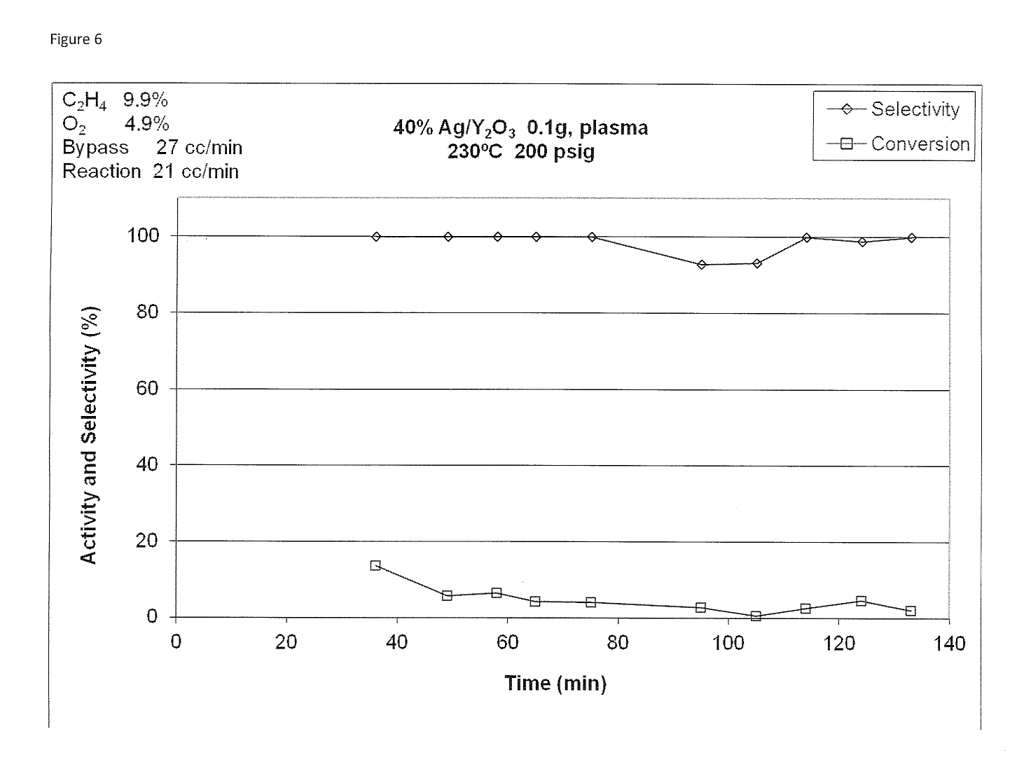

The epoxidation reaction corresponding to FIG. 6 was run using a flow composition with a relatively low ethylene concentration of 9.9 vol % and an oxygen concentration of 4.9 vol % but at a higher pressure of 200 psig. The flow rate was maintained at 12.5 standard cubic centimeters per minute (sccm). The pressure was maintained using a back pressure regulator. FIG. 6 demonstrates a selectivity of greater than about 90% for the duration of the reaction and about 100% for significant portions of the reaction time. Generally, catalyst activities were acceptable.

### Example 3

### Ethylene Epoxidation with Powdered Catalyst: Longer Time Performance

This example demonstrates the superior longer time performance of the catalyst materials described herein in ethylene epoxidation reactions.

To demonstrate long time performance, catalyst particles were formed, and ethylene epoxidation reactions were run, as described in Example 2, above. However, for some of the epoxidation reactions, the carrier gas comprised nitrogen. The catalysts comprised 750 ppm Cs as a promoter metal and some further comprised 40 ppm Re. Specific catalyst and reaction parameters are displayed in Table 2, below.

TABLE 2

| | | | | | Inlet Gas | |
|---|---|---|---|---|---|---|
| | | | | | Composition | |
| | Promotor | Temperature | | Plasma | (vol % C₂H₄, | Carrier |
| FIG. | Composition | (° C.) | Pressure (psig) | Treatment | vol % O₂) | Gas |
| 7 | Cs | 180 | 280 | No | 9.0%, 2.4% | He |
| 8 | Cs | 230 | 200 | After | 9.0%, 3.2% | He |
| | | | | AgNO₃ | | |
| | | | | addition | | |
| 9 | Cs | 230 | 170 | No | 9.0%, 3.2% | He |
| 10 | Cs | 210 | 100-165 | Prior to | 9.0%, 3.0% | He |
| | | | | and After | | |
| | | | | AgNO₃ | | |
| | | | | addition | | |
| 11 | Cs | 180-210 | 270 | After | 9.0%, 3.2% | He |
| | | | | AgNO₃ | | |
| | | | | addition | | |
| 12 | CS | 180 | 200 | Prior to | 9.0%, 3.2% | He |
| | | | | and After | | |
| | | | | AgNO₃ | | |
| | | | | addition | | |
| 13 | Cs and Re | 240-265 | 190 | No | 9.1%, 2.3% | N₂ |
| 14 | Cs and Re | 250-280 | 215-220 | No | 17.4%, 6.1% | N₂ |

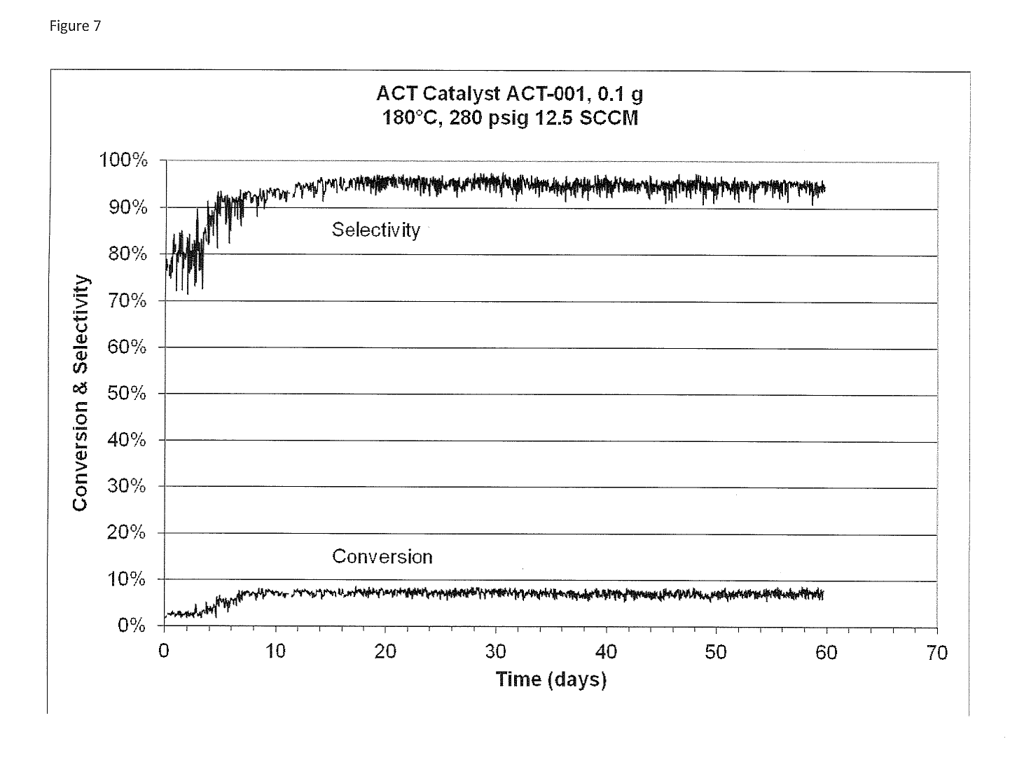

FIG. 7 is a graph containing plots of catalyst conversion and selectivity versus time for a continuous ethylene epoxidation reaction over 60 days. FIG. 7 demonstrates about 95% catalyst selectivity for the duration of the epoxidation reactions after catalyst adjustment to reaction conditions after an initial period of about 12 days. Additionally, reasonable catalyst conversion was observed over the same period. This demonstrates the continued high selectivity of the catalyst materials even after use over many days. It is not clear why the catalyst had an initial period of lower selectivity.

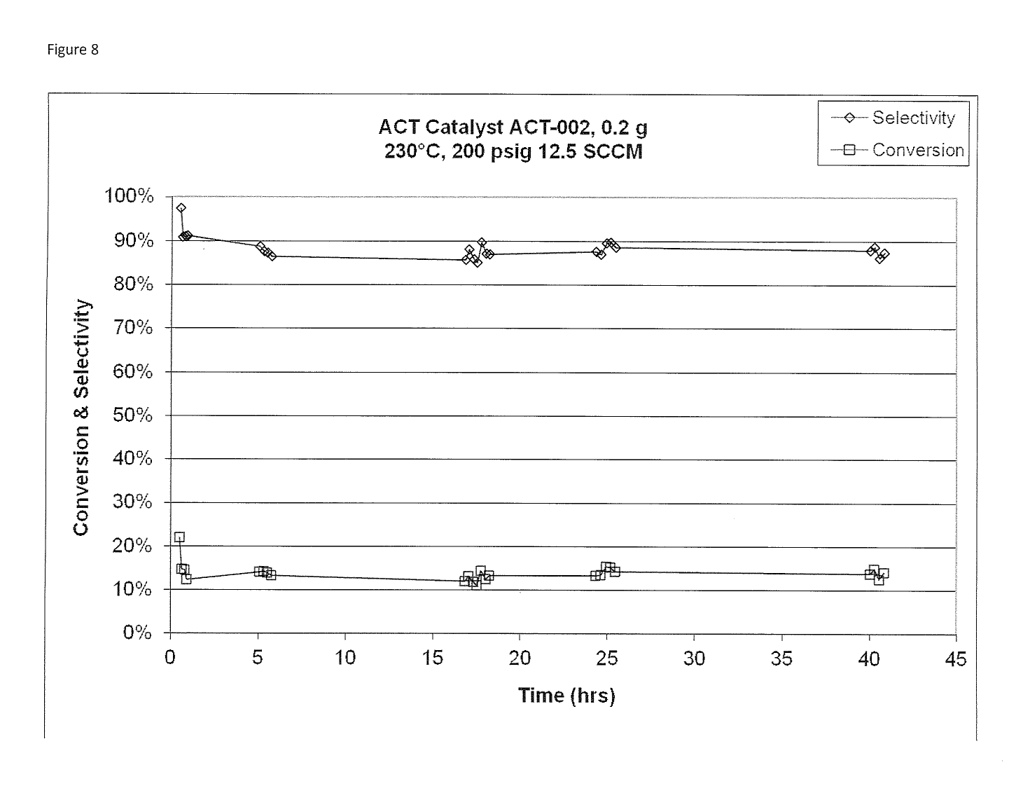

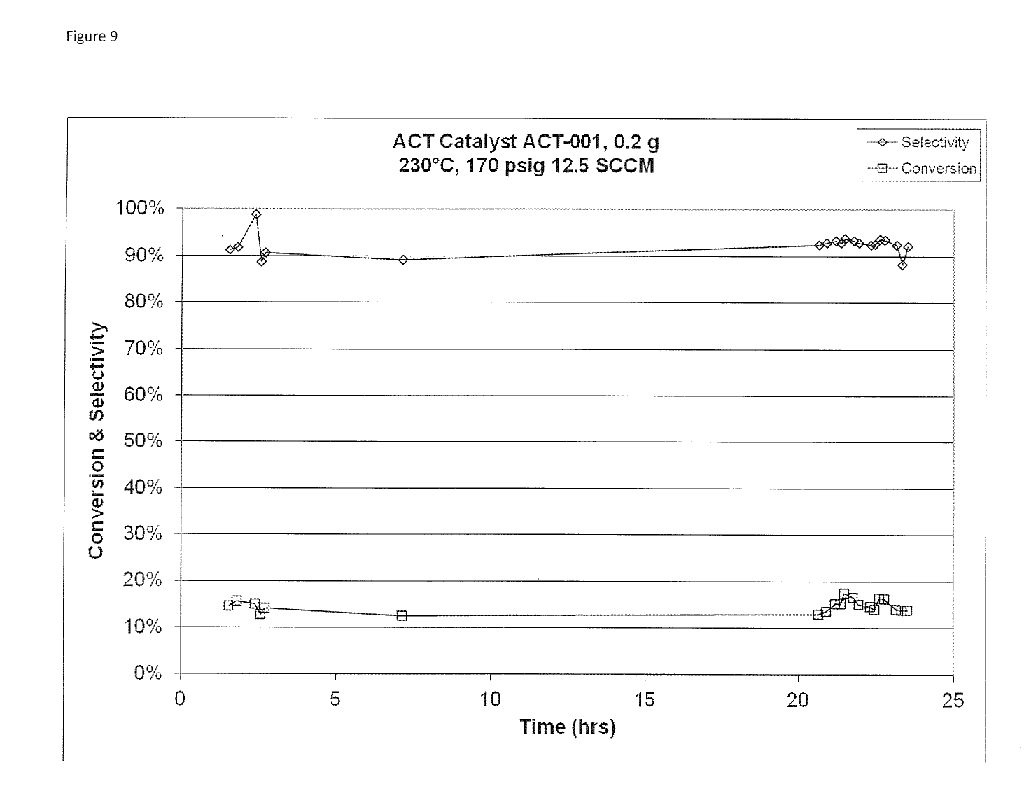

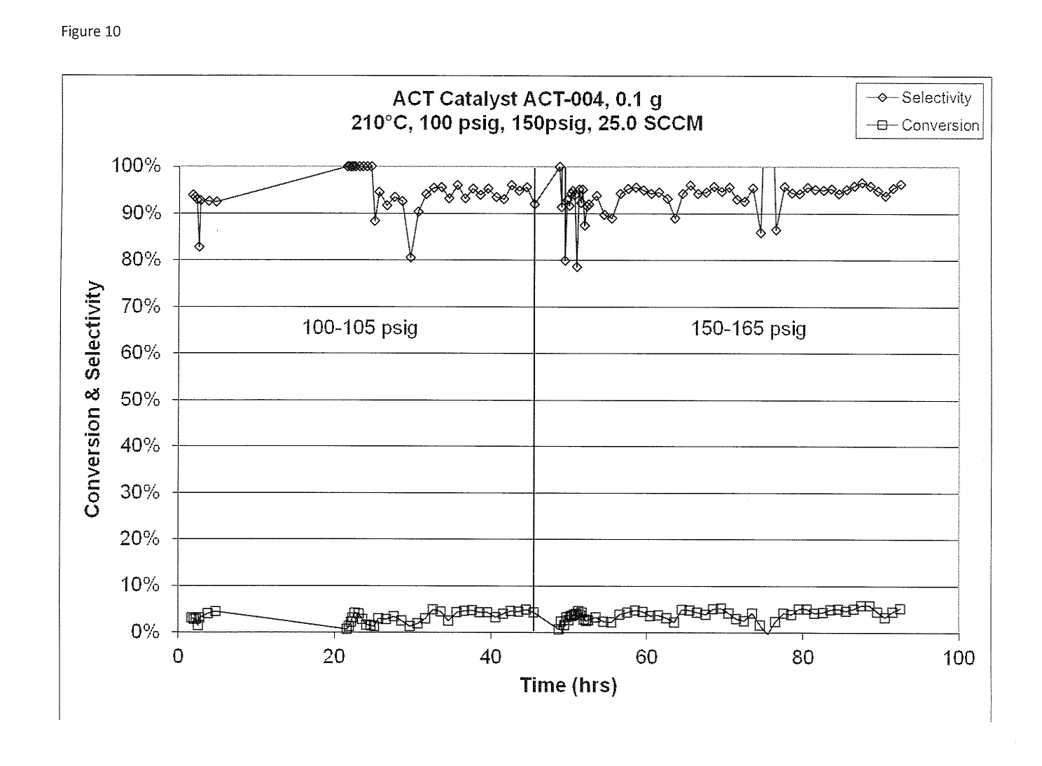

FIGS. 8-10 demonstrate catalyst performance in ethylene epoxidation reactions for a set of catalysts at a few different pressures. The catalyst used in the epoxidation reaction corresponding to FIG. 8 was treated with an oxygen plasma prior to the addition of AgNO₃. The catalysts used in the epoxidation reaction corresponding to FIG. 10 was treated with an oxygen plasma both prior to and after the addition of AgNO₃. The results displayed in FIGS. 8 and 9 were obtained at 200 psig and 170 psig, respectively. The results displayed in FIG. 10 were obtained by initially running the epoxidation reaction at a pressure between 100 psig and 105 psig and subsequently increasing the reaction pressure to between 150 psig and 165 psig after about 46 hours. The flow rates in the experiments in FIGS. 8 and 9 were 12.5 standard cubic centimeters per minute (SCCM) and 25.0 SCCM for the experiments plotted in FIG. 10. Referring to FIGS. 8 and 9, catalyst selectivity was greater than about 85% at higher pressure (FIG. 8) and greater than about 89.5% at the lower pressure (FIG. 9), for the duration of the reactions. Greater selectivities were observed for the experiments plotted in FIG. 10, although the values fluctuated, which may have been related to the greater flow rate. Again, excellent catalyst selectivity and reasonable conversion was observed in all cases.

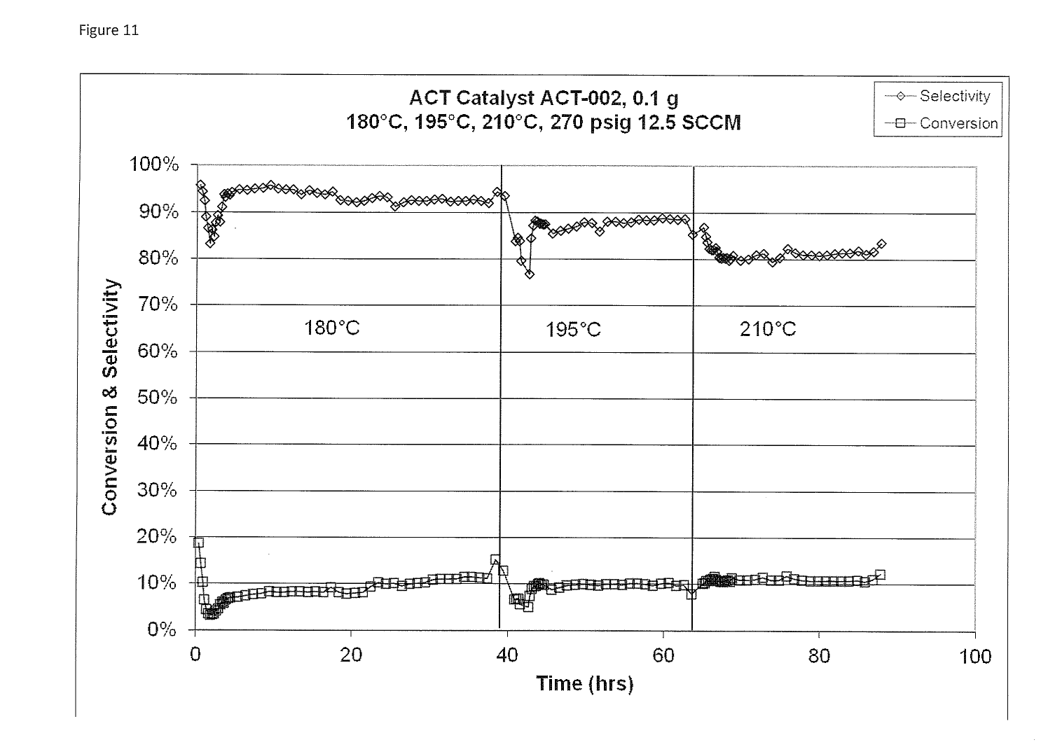

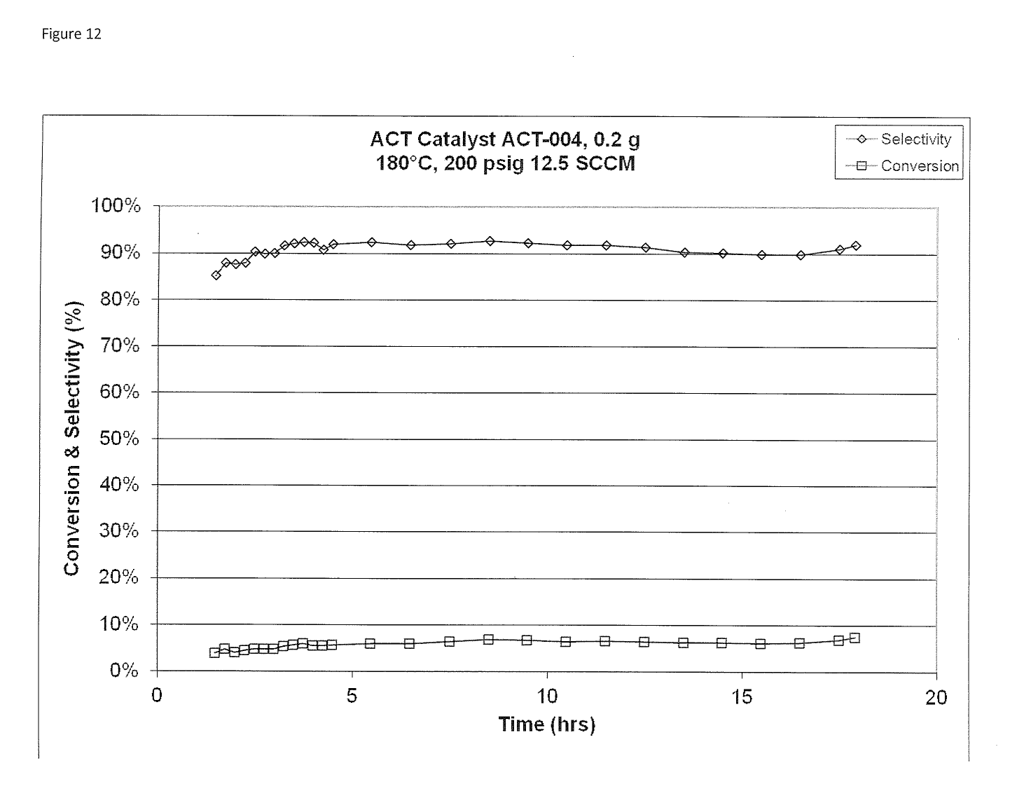

FIGS. 11 and 12 demonstrate catalyst performance in ethylene epoxidation reactions for a set of catalysts at a few different temperatures. The reaction corresponding to FIG. 11 was run at an initial temperature of 180° C. After about 38 hours, the reaction temperature was increased to 195° C. and, subsequently, increased again to about 210° C. after about 64 hours. The reaction corresponding to FIG. 12 was run at 180° C.

Referring to FIG. 11, increasing the reaction temperature generally resulted in decreased catalyst selectivity. In particular, after catalyst adjustment to reaction conditions, catalyst selectivity was greater than about 91% at 180° C. after an initial period, generally between about 85% and 90% at 195° C., and between about 80% and 85% at 210° C. On the other hand, catalyst conversion was substantially the same over the full temperature range tested. Furthermore, comparison of FIGS. 12 and 8 reveals similar behavior of catalyst selectivity with temperature. In particular, FIG. 12 (180° C.) demonstrates catalyst selectivity greater than about 90% after catalyst adjustment to reaction conditions while FIG. 8 (230° C.) reveals catalyst selectivity between about 85% and 90%.

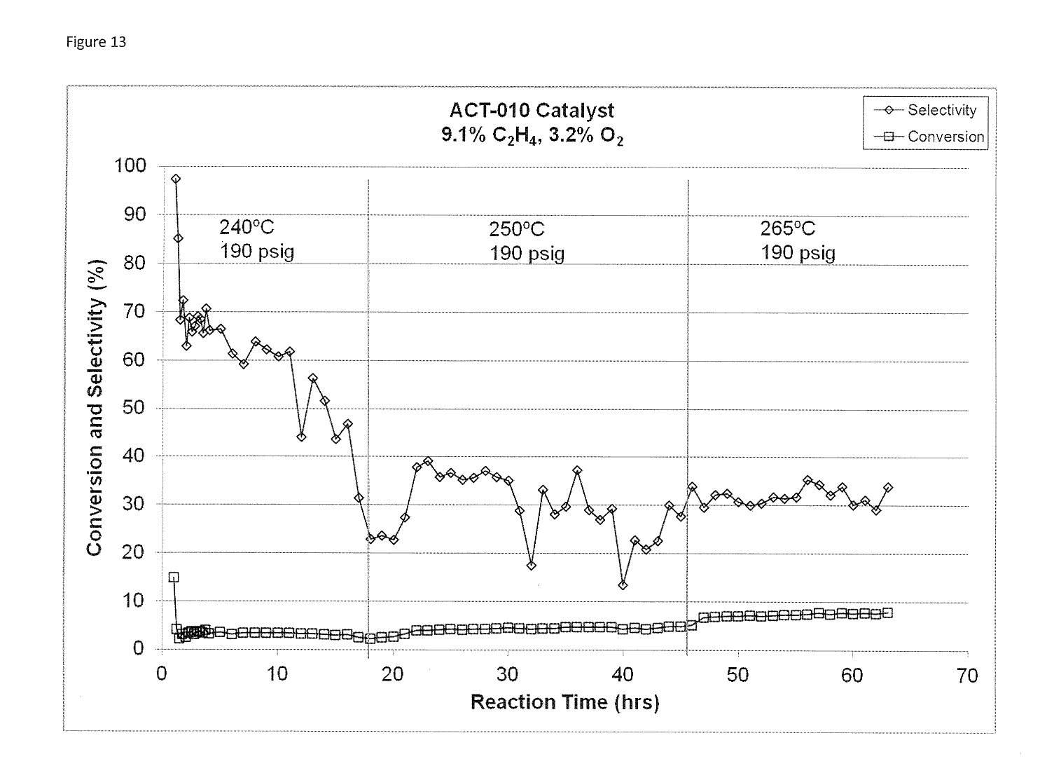

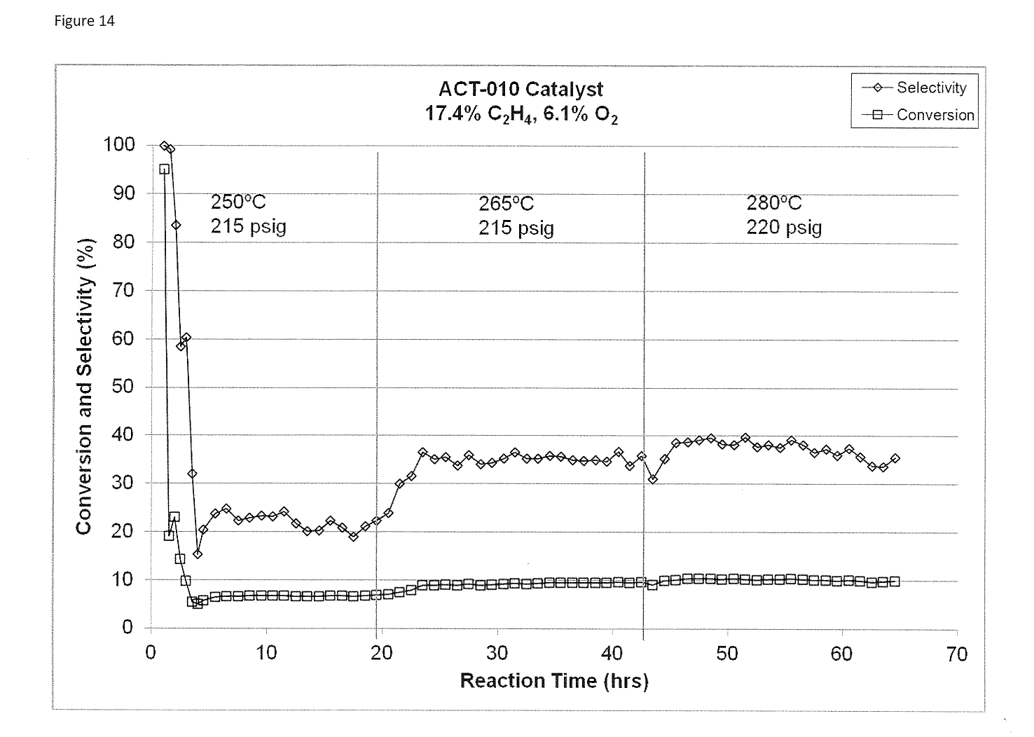

Catalyst performance in ethylene epoxidation reactions with a reactant flow comprising a nitrogen carrier gas is demonstrated in FIGS. 13 and 14. The epoxidation reaction corresponding to FIG. 13 was run at a constant pressure of 190 psig and an initial temperature of about 240° C. After about 19 hours, the temperature was increased to 250° C., and, subsequently, to 265° C. after about 46 hours. The epoxidation reaction corresponding to FIG. 14 was run at pressure between 215 psig and 220 psig and an initial temperature of about 250° C. After about 20 hours, the temperature was increased to 260° C. and, subsequently, to 280° C. after about 43 hours. The reactions performed in nitrogen were performed at higher temperatures than those run in helium to get desired conversion to the product ethylene oxide.

Referring to FIG. 13, catalyst selectivity decreased, and conversion slightly increased, with increasing temperature. The observed temperature dependence of the selectivity and conversion was similar for ethylene epoxidation reactions run with reactant flow comprising a helium carrier gas (see FIG. 11). On the other hand, the catalyst selectivities demonstrated in FIG. 13 are significantly lower than catalyst selectivities observed in ethylene epoxidation reactions at similar temperatures and pressures, but with a helium carrier gas. For example, FIGS. 8 (230° C., 200 psig) and 9 (230° C., 170 psig) demonstrate catalyst selectivities greater than about 85% over the duration of the corresponding epoxidation reactions, significantly greater than the selectivities demonstrated in FIG. 13. Presumably, comparable selectivities can be achieved with the catalyst powders and the nitrogen carrier gases with appropriate adjustment of the reaction parameters.

FIG. 14 reveals that for reactions run at higher pressures and ethylene concentrations, both catalyst selectivity and conversion increased with increasing temperatures. Again, catalyst selectivities were significantly lower than for ethylene epoxidation reactions at similar temperatures and pressures, but with a helium carrier gas.

### Example 4

### Ethylene Epoxidation with Catalyst Pellets

This example demonstrates the performance of the catalyst pellets described herein in ethylene epoxidation reactions.

To demonstrate the performance of catalyst pellets, a catalyst powder was formed as described in Example 1, without plasma treatment. The catalyst powder had approximately 40 weight percent silver and 750 ppm of Cs. Catalyst pellets were then formed from the powder by adding an appropriate amount of the powder to a cylindrical die with a ⅜ inch inner diameter and pressing with a commercial press at about 2000 pounds of force for approximately 10 seconds. The formed catalyst pellets were then fractured with a hammer and chisel to form fragments of pellets that could be used in the tube reactor described above. The fractured catalyst pellet fragments were random shapes ranging from 1 mm-5 mm in any dimension.

The reaction was performed using the glass tube reactor described above in Example 2. The catalyst pellet fragments were held in place in the tube with glass wool and fittings were attached on both ends of the tube to control the flow through the tube. The gas consisted of 17-29 vol % (percent by volume) ethylene as specified more explicitly below and 7-12 vol % oxygen, the remainder of the gas being a carrier gas comprising nitrogen.

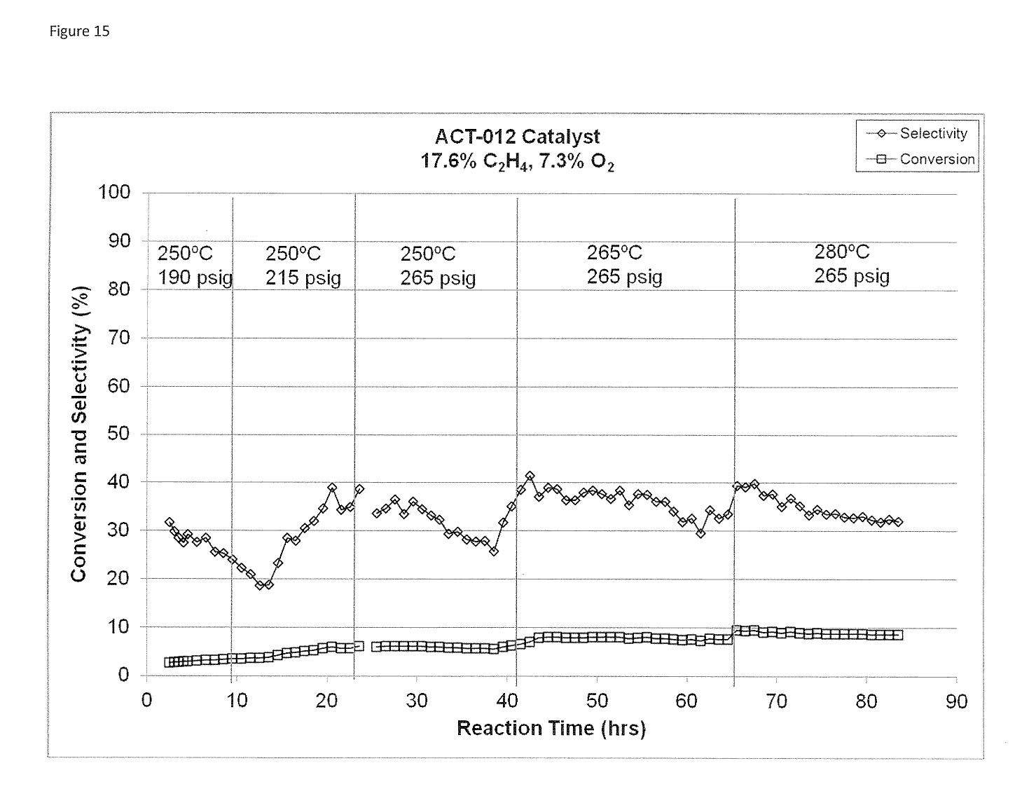

FIG. 15 demonstrates the performance of catalyst pellets at various pressures and temperatures. The reaction corresponding to FIG. 15 was initially run at a temperature of 250° C. and a pressure of 190 psig with a gas comprising 17.6% ethylene and 7.3% oxygen. After about 11 hours, the pressure was increased to 215 psig and, subsequently, increased again to 265 psig after about 24 hours. After about 42 hours, the temperature was increased to 265° C. and, subsequently, increased against to 280° C. after about 65 hours. Comparison between FIGS. 14 and 15 reveal that catalyst pellet performance with respect to conversion and selectivity was similar to catalyst powder performance in similar reaction conditions with N₂ carrier gas.

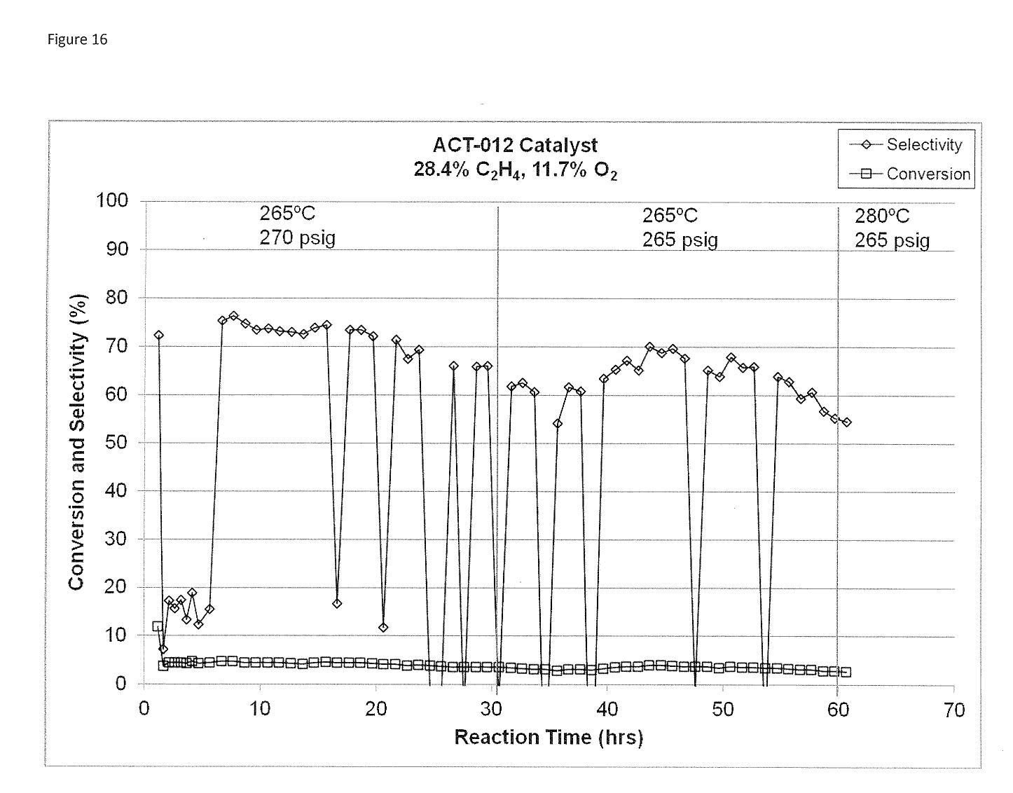

FIG. 16 demonstrates the performance of catalyst pellets at various pressures and temperatures at higher ethylene concentrations. The reaction corresponding to FIG. 16 was initially run at a temperature of about 265° C. and a pressure of about 270 psig with a gas comprising 28.4% ethylene and 11.7% oxygen. After about 31 hours, the reaction pressure was marginally decreased to about 265 psig. After about 59 hours, the reaction temperature was increased to about 280° C. FIG. 16 demonstrates relatively good selectivity and moderate conversion with catalyst pellets. The cause of the spurious oscillations observed in the catalyst selectivity of FIG. 13 was unknown.

The embodiments above are intended to be illustrative and not limiting. Additional embodiments are within the inventive concepts. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein.

What is claimed is:

1. A catalyst pellet comprising a fused particulate material comprising a ceramic material and having a primary particle diameter of no more than about 250 nm, wherein the pellet has a BET surface area of at least about 5 m²/g.

2. The catalyst pellet of claim 1 wherein the pellet density is about 5 percent to about 90 percent of the density of the bulk density.

3. The catalyst pellet of claim 1 further comprising a porous structure wherein the pores have an average diameter from about 1 nm to about 900 nm.

4. The catalyst pellet of claim 1 further comprising an elemental metal coating.

5. The catalyst pellet of claim 1 comprising about 5 weight percent to about 80 weight percent silver as an elemental metal coating.

6. The catalyst pellet of claim 1 having a length and an orthogonal width, wherein the length is no more than about twice the width.

7. A catalyst pellet comprising a fused particulate material comprising an elemental metal having a primary particle diameter of no more than about 250 nm, wherein the pellet has a BET surface area of at least about 5 m²/g.

8. The catalyst pellet of claim 7 wherein the pellet density is about 5 percent to about 90 percent of the density of the bulk density.

9. The catalyst pellet of claim 7 wherein the elemental metal comprises silver.

10. The catalyst pellet of claim 7 having a length and an orthogonal width, wherein the length is no more than about twice the width.

11. A method for forming a nanostructured catalyst pellet, the method comprising pressing in a die a powder comprising a ceramic material with an average primary particle diameter of no more than about 250 nm, an elemental metal or a combination thereof, at a pressure sufficient to fuse the powder into a nanostructured pellet in the shape of the die, the pellet having a BET surface area of at least about 5 m²/g.

12. The method of claim 11 wherein the pressing comprises applying a pressure from about 1000 psi to about 15,000 psi to the powder of particles.

13. The method of claim 11 further comprising heating the particles at a temperature from about 350° C. to about 1000° C.

14. The method of claim 13 wherein the particles are heated for about 2 hours to about 24 hours.

15. A method of preparing a catalyst material, the method comprising:
exposing a material comprising a ceramic material to a low temperature plasma to form a surface treated material; and
depositing elemental metal onto the ceramic material.

16. The method of claim 15 wherein the low temperature plasma is an oxygen plasma or a hydrogen plasma.

17. The method of claim 15 wherein the low temperature plasma is applied for at least about 10 minutes.

18. The method of claim 15 wherein the elemental metal is silver or an alloy thereof.

19. The method of claim 18 wherein the ceramic material comprises yttria.

20. The method of claim 15 wherein the exposing to the low temperature plasma is performed before depositing the elemental metal onto the ceramic material.

21. The method of claim 15 wherein the exposing to the low temperature plasma is performed both before and after depositing the elemental metal onto the ceramic material.

22. A catalyst material comprising at least about 10 weight percent elemental silver and at least about 10 weight percent yttria.

23. The catalyst material of claim 22 having at least 20 weight percent elemental silver.

24. The catalyst material of claim 22 wherein the catalyst is particulate with particles having an average primary particle diameter of no more than about 250 nm.

25. The catalyst material of claim 22 wherein the catalyst comprises yttria particles with particle surfaces coated with the elemental silver.

26. The catalyst material of claim 22 having a BET surface area from about 1 m²/g to about 150 m²/g.

27. The catalyst material of claim 22 further comprising a dopant promoter comprising an alkali metal.

28. The catalyst material of claim 27 wherein the dopant promoter concentration is at least 50 ppm by weight.

29. The catalyst material of claim 27 wherein the dopant promoter comprises Cs.

30. A method for forming ethylene oxide from ethylene, the method comprising contacting ethylene with a catalyst in an atmosphere comprising oxygen, wherein the catalyst has a surface area from about 1 m²/g to about 150 m²/g and wherein the reaction has a selectivity from about 92% to about 100%.

31. The method of claim 30 wherein the reaction has a conversion activity of at least about 5% at about 300° C.

32. The method of claim 30 wherein the reaction is performed at a temperature of no more than about 300° C.

33. The method of claim 30 wherein the catalyst is fixed in a reactor and wherein the contacting of ethylene with the catalyst comprises flowing the ethylene and the oxygen over the catalyst.

34. The method of claim 30 wherein the catalyst comprises at least about 10 weight percent elemental silver and at least about 10 weight percent yttria.

35. The method of claim 30 wherein the catalyst has a BET surface area from about 5 m²/g to about 60 m²/g.

36. The method of claim 30 wherein the selectivity is observed over a period of at least about 2 days.
